(19) United States
(12) Patent Application Publication
(10) Pub. No.: US 2018/0008191 A1
(43) Pub. Date: Jan. 11, 2018

(54) PAIN MANAGEMENT WEARABLE DEVICE

(71) Applicant: KONINKLIJKE PHILIPS N.V., EINDHOVEN (NL)

(72) Inventors: John Cronin, Bonita Springs, FL (US); Aki Sakari Harma, Eindhoven (NL); Nathalie Magali Danielle Dessaud, Wintelre (NL)

(21) Appl. No.: 15/547,565

(22) PCT Filed: Jan. 29, 2016
(86) PCT No.: PCT/EP2016/051857

§ 371 Date: Jul. 31, 2017

Related U.S. Application Data
(60) Provisional application No. 62/110,669, filed on Feb. 2, 2015.

(30) Foreign Application Priority Data
Jul. 7, 2015 (EP) 15175675.6

(51) Int. Cl. A61B 5/00; A61B 5/11; A61B 5/021; A61B 5/01; A61B 5/024; A61B 5/08; A61B 5/0205

(52) U.S. Cl. CPC A61B 5/4824; A61B 5/746; A61B 5/1118; A61B 5/7475; A61B 5/4803; A61B 5/7267; A61B 5/021; A61B 5/0205; A61B 5/024; A61B 5/0816; A61B 5/1112; A61B 5/01; A61B 5/4561; A61B 5/1116

(57) ABSTRACT

A computer implemented method for providing pain management using a wearable device determines a predictive model estimating an intensity level of pain as a function of at least one physiological parameter of a user of the wearable device and at least one activity of the user. The activity of the user includes one or combination of a type of the activity, a level of the activity, a location of the activity, and a duration of the activity. The method determines measurements of physiological and activity sensors of the wearable device to produce values of the physiological parameter and the activity of the user and predicts the intensity level of the pain based on the predictive model and the values of the physiological parameter and the activity of the user. The method executes actions based on the predicted intensity level of pain.

### TECHNICAL FIELD

Various embodiments generally relate to wearable technology. More specifically, but not exclusively, various embodiments relate to pain management using wearable devices.

### BACKGROUND

Wearable technology, wearable devices, or simply “wearables” refer to a new class of electronic systems that can provide ubiquitous data acquisition through a variety of unobtrusive sensors. While the sensors provide information about changes in the environment, human activity or health status, there are significant challenges to the coordination, communication, and computation over the ubiquitously collected data. Furthermore, in order to synthesize the information to create useful knowledge or recommendations to consumer end-users many sources of information complementary and in addition to the collected sensor information are needed. These unconventional combinations of information sources require new designs in the hardware and the software components.

The advantages of the wearable device include its proximity to the user and consistency of its computations. For example, a number of wearable devices, while worn by the user, constantly and continuously monitor user's data and/or vital signs of the user. Such information can be useful in subsequent analysis of condition and behavior of the user and/or can be used for performing an action necessitated by the sensed data.

For example, individuals may experience a variety of different bodily pains for a variety of different reasons at various times of the day. Wearable technology, therefore, could be used to monitor a user who experiences bodily pain. Accordingly, there is a need of a wearable device that can help users to manage their pain.

### SUMMARY

Some embodiments are based on realization that wearable devices can be configured to monitor and/or predict pain experienced by a user of a wearable device, predict future occurrences of the pain and/or assist in recognizing the causes of the pain. As used herein, the term “wearable” broadly encompasses devices associated with the user, e.g. worn over or attached to a body part, or embedded into an item of clothing or footwear, and configured for either contact or non-contact sensing of various physiological parameters and activities of the user.

Some embodiments are based on recognition that the same pain symptoms experienced by a user can be caused by different combinations of different reasons. For example, the back pain can be caused by stress (which level could be objectively evaluated by the measurement health rate variability), problems with the spine or can just be a result of sleeping on an old mattress or sitting in an uncomfortable position. To that end, some embodiments are based on realization that the pain experienced by the user needs to be determined not only based on the physiological parameters, but also based on other activities of the user.

As used herein, the physiological parameter can include, but are not limited to various vital signs of the user, such as hydration, calories, blood pressure, blood sugar, blood glucose, insulin, body temperature, heat, heat flux, heart rate, weight, sleep pattern, number of steps, velocity, acceleration, vitamin levels, respiratory rate, heart sound, breathing sound, movement speed, skin moisture, sweat detection, sweat composition, or nerve firings of the user. The physiological parameters can be determined, e.g., measured by a physiological sensor. For example, photoplethysmography (PPG) or bioimpedance measurements can serve as an indication of intensity level of pain experience by the user, as markers of a reaction of the sympathetic nervous system.

In contrast with the physiological parameters that usually can be directly measured, in some situations, the activity of the user needs to be inferred based on the other measurements and/or inputs from the user. For example, using various combinations of measurements of the location, time of the day, heart rate, and an acceleration of the user, some embodiments can determine whether a user is running in a park or at the gym, driving a car to work, sleeping in a bedroom or sitting in an office. To that end, the activity of the user can be defined by one or combination of a type of the activity, a location of the activity, and duration of the activity. Some embodiments use a function of at least one physiological parameter and at least one activity of the user to predict the pain of the user and/or to determine the cause for the pain experienced by the user.

Various embodiments are directed to a computer implemented method directed towards a pain management wearable device including determining a predictive model estimating an intensity level of pain as a function of at least one physiological parameter of a user of the wearable device and at least one activity of the user, wherein the activity of the user includes one or combination of a type of the activity, a level of the activity, a location of the activity, and a duration of the activity; determining concurrently measurements of one or more physiological sensors of the wearable device and one or more activity sensors of the wearable device to produce values of the physiological parameter and the activity of the user; predicting the intensity level of the pain based on the predictive model and the values of the physiological parameter and the activity of the user; and executing one or more actions based on the predicted intensity level of pain.

In a further embodiment, the determining the predictive model comprises obtaining inputs from the user via a user interface, the inputs corresponding to the intensity level of an occurrence of pain experienced by the user; obtaining physiological data from the physiological sensors; obtaining activity data from the activity sensors including one or combination of at least one motion sensor for determining the type of the activity and at least one location sensor for determining the location of the activity; wherein the physiological data and the activity data are taken concurrently with the occurrence of pain experienced by the user; and correlating the intensity levels of the occurrence of pain experienced by the user with the obtained physiological data and the activity data to determine the predictive model.

In a further embodiment, the user interface includes a microphone for accepting auditory inputs, further comprising: classifying the auditory inputs to determine the intensity level of the occurrence of pain experienced by the user.

In a further embodiment, the executing comprises evaluating the predicted intensity level of pain with rules stored in a memory of the wearable device; and executing one or more actions based on the evaluation of the predicted intensity level of pain with the rules.

In a further embodiment, the determining the predictive model comprises obtaining physiological data from the measurements of the physiological sensors collected for a period of time; obtaining activity data from the measurements of the activity sensors collected for the period of time; obtaining times and intensity levels of occurrences of pain experienced by the user within the period of time; and determining the predictive model as a regression function correlating different intensity levels of pain with combinations of the physiological data and the activity data.

In a further embodiment, the regression function is a multi-dimensional function, wherein a particular dimension of the regression function corresponds to values of a particular physiological parameter or a particular activity of the user.

In a further embodiment, wherein the predicted intensity level of pain is above a threshold, further comprising determining sensitivities of the regression function at a point corresponding to the values of the physiological parameter and the activity of the user along at least some dimensions of the regression function; determining a dimension of the regression function with the highest sensitivity leading to a decrease of the intensity levels of pain on the regression function; and executing the action that commands to modify the values of the physiological parameter or the activity of the user corresponding to the dimension.

In a further embodiment, wherein the predicted intensity level of pain is below a threshold, further comprising: determining sensitivities of the regression function at a point corresponding to the values of the physiological parameter and the activity of the user along at least some dimensions of the regression function; determining a dimension of the regression function with the highest sensitivity leading to an increase of the intensity levels of pain on the regression function to above the threshold; and executing the action that commands to modify the values of the physiological parameter or the activity of the user corresponding to the dimension.

In a further embodiment, the method includes updating the predictive model in response to receiving the time and the intensity level of the occurrence of pain experienced by the user.

In a further embodiment, the method further includes receiving a time instance and the intensity level of the occurrence of pain experienced by the user at the time instance; retrieving a subset of the physiological data and the activity data of the user preceding the time instance; and updating the predictive model using the subset of the physiological data and the activity data and the intensity level of the occurrence of pain experienced by the user at the time instance.

Various embodiments are directed to a wearable device for providing pain management including a user interface configured for obtaining inputs from a user of the wearable device, each input indicates an intensity level of an occurrence of pain experienced by the user; one or more physiological sensors to measure a value at least one physiological parameter of the user; one or more activity sensors to determine a value of at least one activity of the user, wherein the activity of the user includes one or combination of a type of the activity and a location of the activity; and a processor executing instructions stored in memory, wherein the processor is configured for executing the instructions to: determine a predictive model estimating an intensity level of a pain as a function of the physiological parameter of the user of the wearable device and the activity of the user; predict the intensity level of the pain based on the predictive model and the values of the physiological parameter and the activity of the user obtained from the physiological and the activity sensors; and execute one or more actions based on the predicted intensity level of pain.

In a further embodiment, the user interface includes a microphone.

In a further embodiment, the processor determines the predictive model by: obtaining physiological data from the measurements of the physiological sensors collected for a period of time, wherein the physiological data is time-series data; obtaining activity data from the measurements of the activity sensors collected for the period of time, wherein the activity data is time-series data; obtaining times and intensity levels of occurrences of pain experienced by the user within the period of time; and determining the predictive model as a regression model correlating different intensity levels of pain with a time series profile formed by a combination of the physiological data and the activity data.

In a further embodiment, wherein at least one action executed based on the evaluation includes notifying the user by displaying a message on the wearable device.

Various embodiments are directed to a non-transitory computer-readable storage media having embodied thereon a program executable by a processor to perform a method for providing pain management using a wearable device including executable instructions for determining measurements of one or more physiological sensors of the wearable device to produce a value of a physiological parameter of a user of the wearable device; determining measurements of one or more activity sensors of the wearable device to produce a value of an activity of the user of the wearable device, wherein the activity of the user includes one or combination of a type of the activity and a location of the activity; predicting the intensity level of pain based the values of the physiological parameter and the activity of the user and a predictive model correlating an intensity level of a pain as a function of at least one physiological parameter of the user and at least one activity of the user; and executing one or more actions based on the predicted intensity level of pain.

The embodiments described above and herein may help to record a level of bodily pain the user experiences along with obtaining sensor data to monitor and track when the bodily pain arises and then subsequently use the sensor data to predict when the bodily pain may resurface. The prediction can be used to provide information as to how the user may mitigate the bodily pain before the pain occurs.

Preferred embodiments of the invention are defined in the dependent claims. It should be understood that the claimed device and the non-transitory computer-readable storage medium can have similar preferred embodiments and the corresponding advantages as the claimed method and as defined in the dependent method claims.

### BRIEF DESCRIPTION OF THE DRAWINGS

FIG. 1 illustrates an exemplary system for pain prediction.

FIG. 2 illustrates exemplary graphical displays that quantify pain experienced by the user.

FIG. 3 illustrates exemplary wearable device pain management software as described herein.

FIG. 4 illustrates exemplary wearable pain management GUI.

FIG. 5A illustrates an exemplary method for the base software of the pain management wearable device.

FIG. 5B illustrates a block diagram of a system for recognizing different level of pains from auditory inputs.

FIG. 6 illustrates an exemplary computing device architecture that may be utilized to implement the various features and processes described herein.

FIG. 7 illustrates an exemplary method for training a microphone.

FIG. 8A illustrates an exemplary method for the wearable sensor software.

FIG. 8B illustrates an exemplary method for the subjective pain level software.

FIG. 9 illustrates an exemplary subjective pain level GUI.

FIG. 10A illustrates an exemplary situation for the wearable sensors software.

FIG. 10B illustrates an exemplary rules database.

FIG. 11 illustrates an exemplary receiver GUI.

FIG. 12A illustrates an exemplary long-term history database.

FIG. 12B illustrates an exemplary context database.

FIG. 13 illustrates an exemplary overall method for pain prediction.

FIG. 14 shows a block diagram of a method for providing the pain management using a wearable device according to some embodiments.

FIGS. 15A, 15B, and 15C illustrate different combinations of the physiological parameters and activities of the user used for determining a predictive model according to different embodiments.

FIG. 16 illustrates a block diagram of a method for determining the predictive model according to some embodiments.

FIG. 17 illustrates an exemplar table showing different combinations of the physiological parameters and activities of the user used for determining the predictive model according to some embodiments.

FIG. 18 illustrates a schematic of training a regression function according to some embodiments.

FIG. 19 illustrates a block diagram of a method for predicting the future pain and/or for determining the cause of the pain according to some embodiments.

### DETAILED DESCRIPTION

Various embodiments are directed towards systems and methods for predicting future occurrences of pain. The predictions for future occurrences of pain are based on sensor data and user input of past occurrences of pain. For example, a wearable device may be incorporated to monitor conditions and/or parameters that may be associated with occurrences of pain. In some embodiments, the systems and methods additionally or alternatively detect the onset of pain and, based on past occurrences and other information about the current context, predict the duration of the pain (e.g., a few minutes or a few hours). The user may also provide input regarding the occurrences of pain. The information about the past occurrences of pain may be evaluated to predict future occurrences of pain similar to the occurrences that have happened in the past. In this way, the user can take various precautions in view of the predictions in order to mitigate effects of experienced pain when the pain actually occurs.

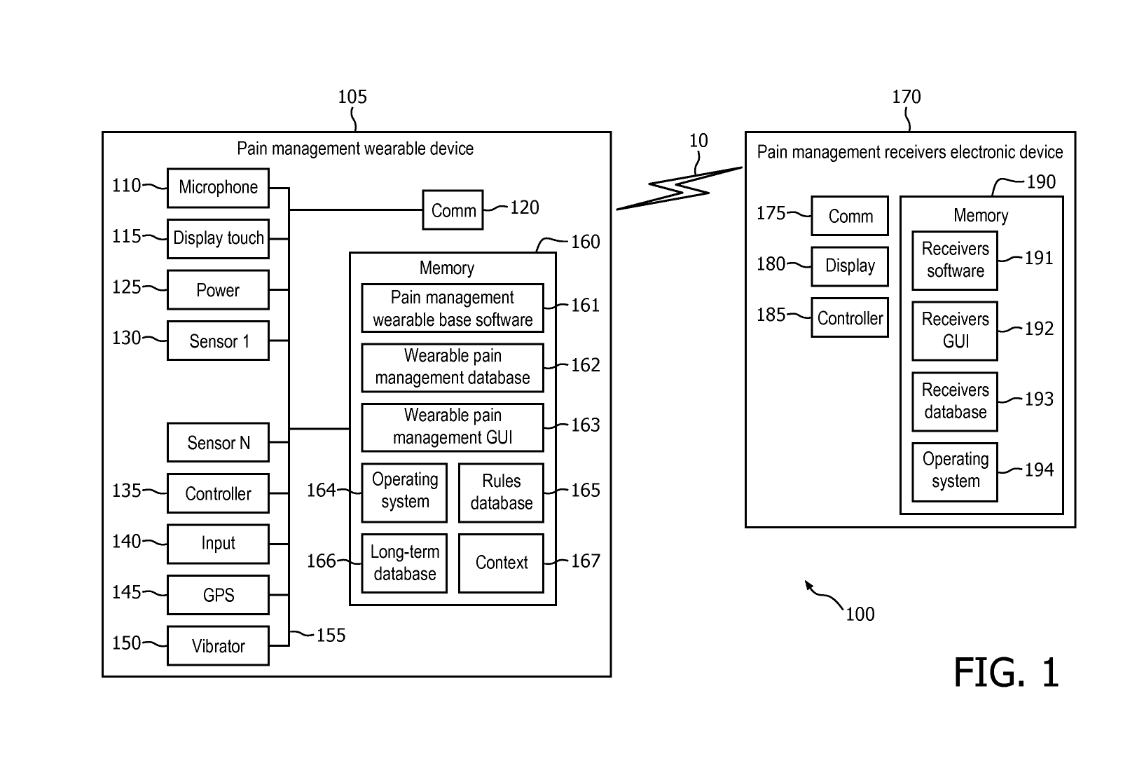

FIG. 1 illustrates an exemplary system 100 for pain prediction. In particular, the system 100 may include two devices: a pain management wearable device 105, and a pain management receiver electronic device 170.

The pain management wearable device 105 may be worn on the body of the user (e.g., arm, wrist, chest, etc.). As illustrated in the figure, the pain management wearable device 105 may include various different elements. These elements may include a microphone 110, a display 115, communication module 120, power supply 125, a plurality of sensors (1−N) 130, a controller, 135, input elements 140, a global positioning system (GPS) element 145, a vibrator 155 and memory 160. It should be noted that these elements of the pain wearable device 105 may all be connected to a central bus 155. As used herein, the central bus 155 may be used to transfer data between the various elements of the pain management wearable device 105. The central bus 155 may include related hardware components (e.g., wire, optical fiber) and software (e.g., communication protocols).

The microphone 110 may be used by the pain management wearable device 105 to receive input about an experience of pain from the user. These inputs are indicative of subjective level of pain of the user. The input received by the microphone 110 is in the form of pain sounds, such as groan, grunt, etc.

The pain management wearable device 105 may also include one or more input elements 140. The input elements 140 may be incorporated with the pain management wearable device 105 to facilitate user input of information (e.g., subjective pain levels) into the wearable device. The subjective pain levels may be provided in form of numerical input, such as on a scale of 1-10. The subjective pain levels may be also provided in form of broader indication, such low, medium, and high. The input elements 140 may include, for example, buttons, wheel, or a switch. The user may utilize these input elements 140 when interacting with, for example, a graphical user interface (GUI) displayed on the pain management wearable device 105. These input elements 140 may facilitate the user, for example, in selecting one or more various options displayed on the GUI.

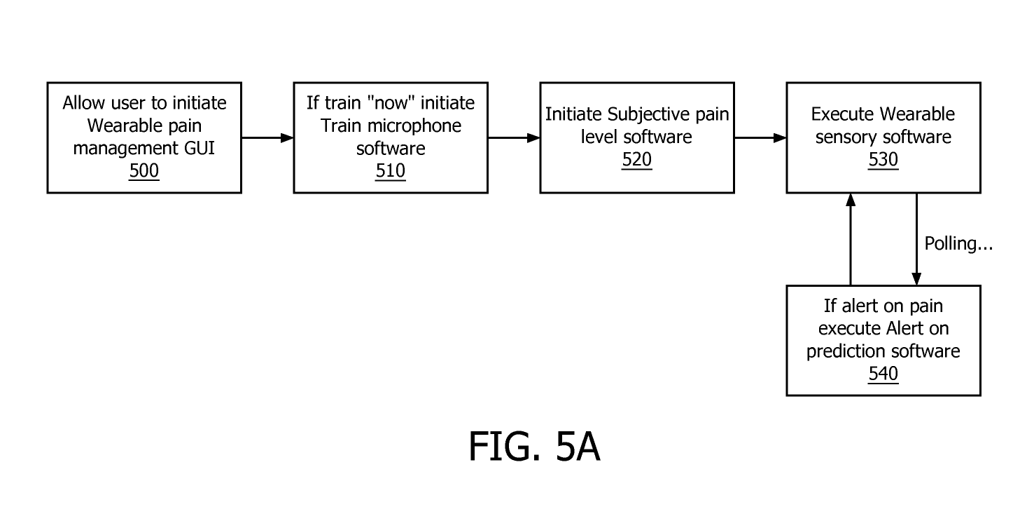

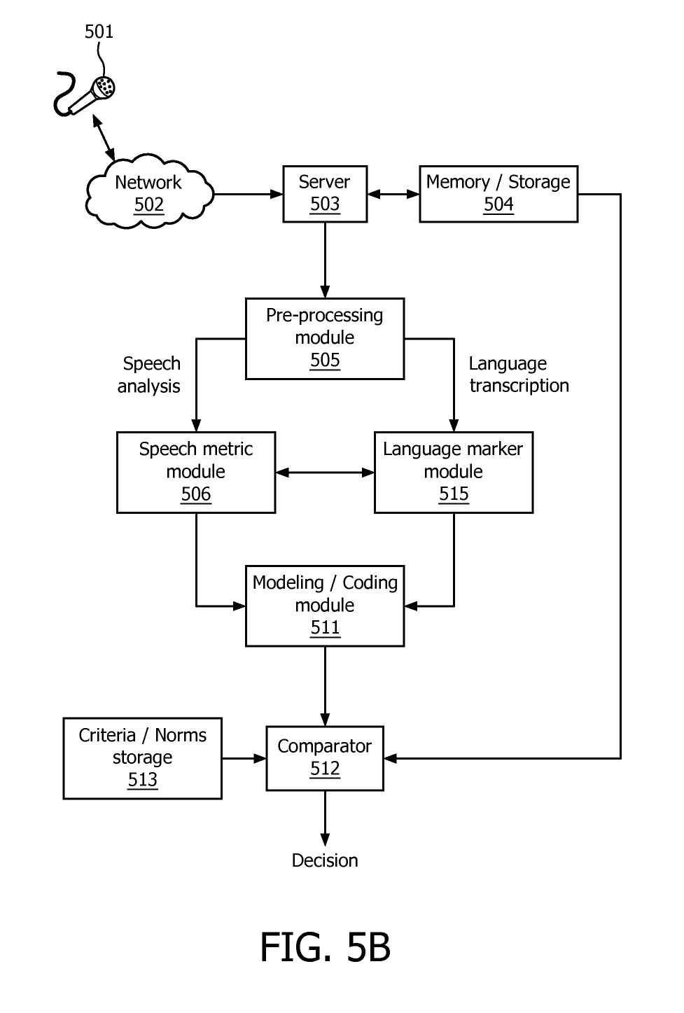

Thus, subjective level of pain may be provided by a microphone 110 or input element 140. These inputs can be used to train the wearable device 105 for prediction of the onset/likelihood of pain. This will be explained later in conjunction with FIG. 5.

The pain management wearable device 105 may also include a display 115. The display 115 may be used by the pain management wearable device 105 to display various types of information or facilitate interaction between the user and the pain management wearable device 105 (e.g., GUI). In some embodiments, the display 115 may also be a touchscreen display that may allow the user to directly interact (e.g., provide input via input elements 140) with the wearable device through physical contact with the display 115.

The communication module 120 may facilitate communication (e.g., wireless communication) between the pain management wearable device 105 and other devices (e.g., wearable devices, smart devices) and/or networks. For example, as illustrated in FIG. 1, the communication module 120 may facilitate communication 10 (e.g. wired or wireless) with the pain management receivers electronic device 170. The communication module 120 may implement the communication through the use of one or more methods known in the art including Wi-Fi, Bluetooth, 3G, 4G, LTE, near field communication (NFC).

The power supply 125 may be included to provide power for the operation of the pain management wearable device 105. The power supply 125 may be implemented through the use of a capacitor or a battery. The power supply 125 may also be capable of being charged or re-charged using an external power source (e.g., battery charger).

The pain management wearable device 105 may include a plurality of sensors 130. The sensors 130 may be included to measure different parameters (e.g., environmental conditions, physiological parameters) related to an experience of pain of the user. For example, the sensors can include physiological sensors for obtaining physiological data and activity sensors for obtaining activity data. For example, the activity sensors can include one or combination of a motion sensor for determining the type of the activity and a location sensor for determining the location of the activity. For example, the physiological and activity sensors can determine various vital signs of the user, such as hydration, calories, blood pressure, blood sugar, blood glucose, insulin, body temperature, heat, heat flux, heart rate, weight, sleep pattern, number of steps, velocity, acceleration, vitamin levels, respiratory rate, heart sound, breathing sound, movement speed, skin moisture, sweat detection, sweat composition, or nerve firings of the user. Different types of measurements and/or sensors can be used for determining the same parameter. For example, Heart rate can be measured via photoplethysmography (PPG), and the level of stress can be estimated from heart rate variability measured by PPG or by skin conductance using bioimpedance measurements. Similarly, the location of the user can be determining using an accelerometer and/or global positioning system (GPS).

The sensor data obtained may be related to the particular experience of pain and can be therefore be used to predict future occurrences of similar pain. For example, sensor data can measure pain by measuring a current blood pressure or temperature at the time the user is experiencing pain. In another situation, sensor data may also be used to monitor movement of the user and match the sensor data with subjective levels of pain provided by the user. Use of this matching may be helpful in monitoring the movement of the user while they are recovering, for example, from broken bones and informing the user what movements may be allowed.

It should be noted that the sensor data may be used in many other ways as well. For example, the sensor data may also be helpful in evaluating an actual level of intensity corresponding to an experience of pain. The sensor data may also be used to determine a frequency of repeat occurrences for the corresponding experience of pain.

The processor/controller 135 of the pain management wearable device 105 may be any computer processor known in the art. The processor/controller 135 can be used to carry out the various instructions of the pain management wearable device 105 (e.g., analysis of sensor data, calculations). In some embodiments, the pain management wearable device 105 may include two or more processors/controllers.

The GPS element 145 may be used by the pain management wearable device 105 in order to determine a physical location of the user. The physical location may be beneficial in evaluating whether the location of the user impacts the experience of pain. The context of where the pain is experienced (e.g., work, home, in the car) may be obtained by the GPS and stored in memory of the pain management wearable device 105. The context data may be utilized by the pain management wearable device 105 when making predictions alongside sensor data.

As noted above, the vibrator 150 may also be included in the pain management wearable device 105. The vibrator 150 may be used, for example, as a way for the pain management wearable device 105 to notify the user. In some embodiments, the pain management wearable device 105 may instruct the vibrator 150 to vibrate in situations where pain is predicted to occur soon, for example, based on changing environments or user participation in a particular activity.

The memory 160 of the pain management wearable device 105 may be used to store data associated with the pain management wearable device 105. It should be noted that the memory 160 may also include various other software and databases for carrying out the functionality of the pain management wearable device 105. As illustrated in FIG. 1, the memory 160 may include pain management wearable base software 161, a wearable pain management database 162, a wearable pain management GUI 163, an operating system (OS) 164, rules database 165, a long-term database 166, and a context database 167.

The pain management wearable base software 161 of the pain management wearable device 105 may be responsible for the management and operation of the pain management wearable device 105. In some embodiments, the pain management wearable base software 161 may poll for sensor data relating to an exposure level for the user. The pain management wearable base software 161 may also execute software and other elements within the pain management wearable device 105 to carry out the functionality of the pain management wearable device 105. For example, the pain management wearable base software 161 may instruct the pain management wearable device 105 to obtain sensor data from one or more sensors 130. In another example, the pain management wearable base software 161 may execute a process for training and using the microphone. Further discussion of the pain management wearable base software can be seen below (see FIG. 5).

The pain management database 162 may be used to store information obtained by the pain management wearable device 105. For example, sensor data obtained by the plurality of sensors 130 and inputs from the user related to experienced pain may all be organized and stored within the pain management wearable device 105. It should be noted that other types of information obtained and generated by the pain management wearable device 105 may also be stored within the wearable pain management database 162.

In some embodiments, the pain management database 162 stores the predictive model for estimating intensity level of pain of the user as a function of at least one physiological parameter of a user of the wearable device and at least one activity of the user. In one embodiment, the predictive model is trained in advance by correlating the intensity levels of occurrences of pain experienced by the user with physiological and activity data of the user.

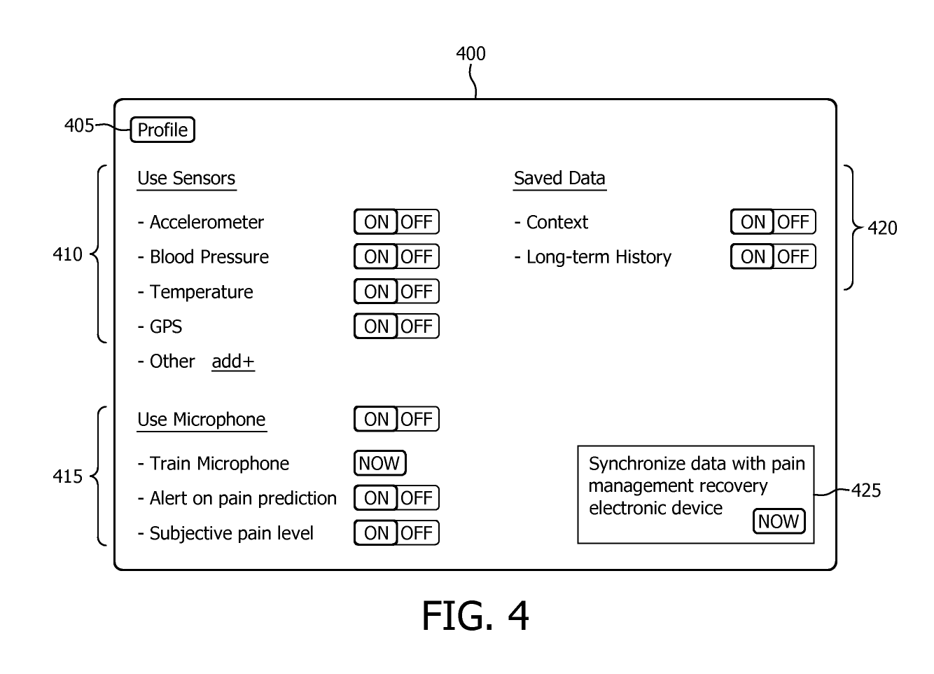

The pain management GUI 163 may be used by the user to manage and customize operation of the pain management wearable device 105. The pain management GUI 163 may be displayed, for example, on the display 115 of the pain management wearable device 105 for the user to interact with. As noted above, the user may be able to provide inputs using one or more input elements 140. In another embodiment, the display 115 may be touch-based. A touch-based display may allow the user to interact with the various elements of the pain management GUI 163 directly. Additional information about the pain management GUI 163 is provided below with respect to subject matter related to FIG. 4.

The OS 164 is software that can be used to manage the various elements and resources associated with the pain management wearable device 105. Exemplary OS 164 that may be used with the pain management wearable device 105 include Darwin, RTXC, LINUX, UNIX, OS X, ANDROID, WINDOWS, or an embedded operating system such as VxWorks.

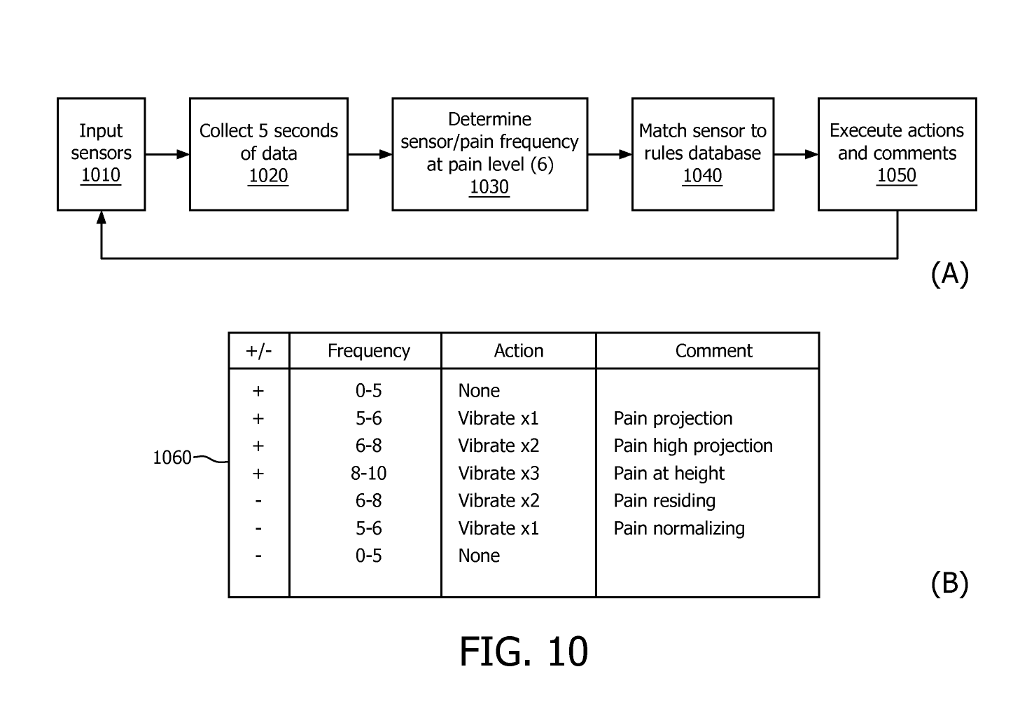

The rules database 165 stores rules or guidelines that can assist the user in mitigating or preventing the pain in current or future situations when pain may be experienced by the user. For example, if a user is experiencing an increasing intensity in the amount of pain experienced with each occurrence over a period of time, a rule can be stored in the rules database that takes this information stored in memory (e.g., wearable pain management database 162) in order to instruct the pain management wearable device 105 to notify the user that in these situations that the experienced level of pain is increasing. The alert may also provide suggestions to mitigate the experienced pain or provide a message, for example, informing the user of one or more ways the user can mitigate the pain. The message may also inform the user that medical assistance may be required. In some embodiments, other types of rules may also be stored within the rules database 165. For example, rules may also be directed at monitoring situations when the pain being experienced by the user is subsiding. Such information may be beneficial in providing a notice, for example, that some medication, treatment, or action being performed by the user to mitigate the experienced pain is working. Further details are provided below in FIG. 10B.

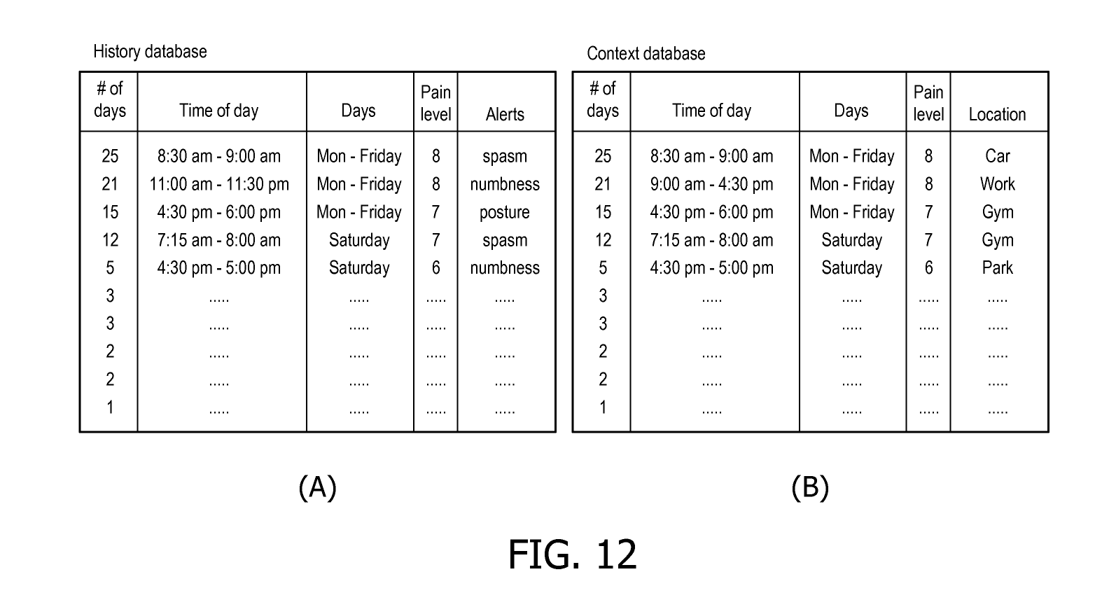

The memory 160 may also include a long-term database 166. Whereas the information stored in the wearable pain management database 162 may be continually updated, for example, with recently acquired sensor data, the long term-database 166 may continually accumulate the sensor data over a long period of time. In this way, the long-term data may be used to evaluate, for example, whether the health condition of the user is becoming better or worse over time. The long-term data may also be used for pain prediction. Further details regarding the long-term database 162 can be found below with respect to FIG. 12A.

The context database 167 may store the GPS-based data obtained, for example, by the GPS element 145. The user may also be able to provide input identifying a location through the use of the pain management wearable device 105. As noted above, location-based data (which may be part of context data) may also influence situations where the user experiences pain. For example, the user may experience pain in their lower back if they are slouching in their chair at work. The user may also experience pain while participating in one or more activities at the gym. These are only some examples of how context data may be used in pain prediction. Further details regarding the context database 167 can be found below with respect to FIG. 12B.

Also illustrated in FIG. 1 is the pain management receiver electronic device 170. The pain management receiver electronic device 170 may be implemented, for example, using a smart device such as a laptop, desktop, tablet, or mobile device.

The pain management receiver electronic device may also include a number of different elements. These elements may include a communication module 175, a display 180, a controller 185 and memory 190.

The communication module 175, the display 180 and the controller 185 may be similar to the communication module 120, the display 115 and the controller 135 described above with the pain management wearable device 150. The memory 190 of the pain management receiver electronic device 170, however, may include different elements. As illustrated in the figure, the memory 170 may include receiver software 191, receiver GUI 192, receiver database 193 and an OS 194.

The receiver software 191 may be directed at facilitating synchronization of the data stored in the pain management wearable device 105 with the pain management receiver electronic device 170. In particular, the receiver software 191 may operate in conjunction with the receiver GUI 142.

The receiver GUI 142 may be used by the pain management receivers electronic device 170 to provide reports for the user to view on the display of the pain management receiver electronic device 170. These reports may include information obtained by the pain management wearable device 105 related to pain intensity, pain frequency, and user subjective inputs about each occurrence of pain.

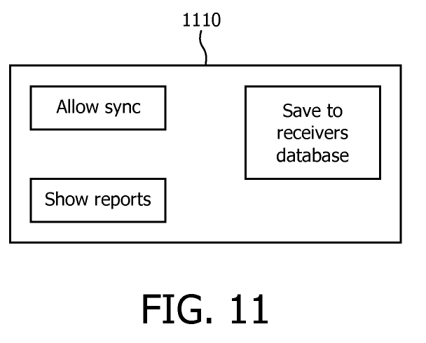

It should be noted that the receiver GUI 192 may facilitate in the synchronization of information (e.g., sensor data) between the pain management wearable device 105 with the pain management receiver electronic device 170. The receiver GUI 192 may also provide reports to the user that includes information such as pain intensity, pain frequency and corresponding subjective inputs from the user with each occurrence of pain. Further details are provided below in FIG. 11.

The reports displayed by the receivers GUI 192 may be stored in memory 190 of the pain management receivers electronic device 170. Furthermore, any information obtained from the pain management wearable device 105 may be organized and stored in the receiver database 193 as well.

Similarly raised above with the OS 164 of the pain management wearable device 105, the OS 194 of the pain management receivers electronic device 170 may be included for the same functions. The OS 194 is software that can be used to manage the various elements and resources associated with the pain management receivers electronic device 170. Exemplary OS 194 that may be used with the pain management receiver electronic device 170 may also include Darwin, RTXC, LINUX, UNIX, OS X, ANDROID, WINDOWS, or an embedded operating system such as VxWorks.

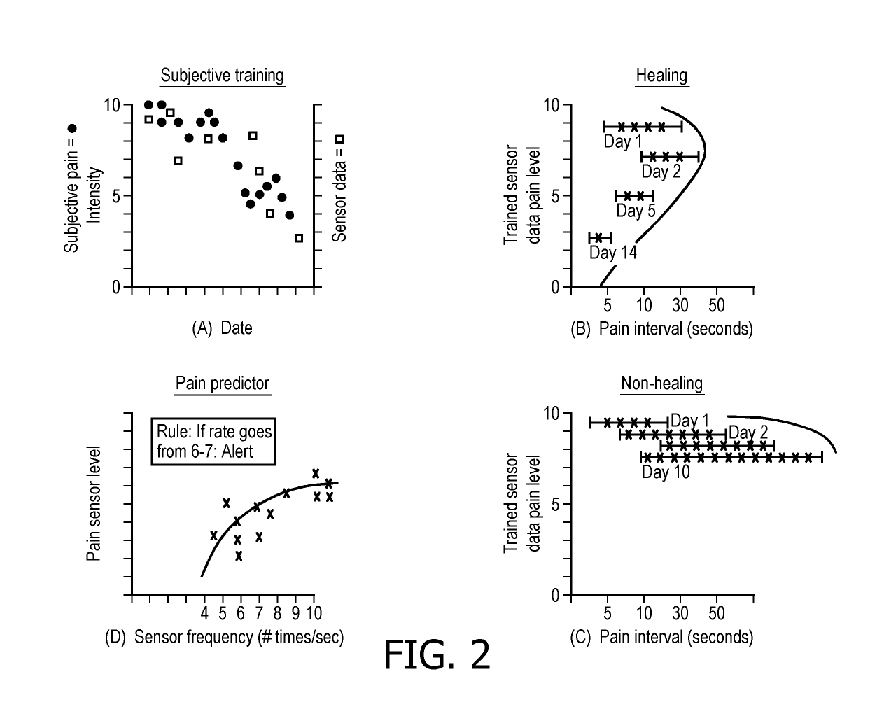

FIG. 2 illustrates exemplary correlation between intensities of the pain experienced by the user and various measurements of the sensor of the wearable device. In particular, FIG. 2 illustrates four different graphical plots representing features of pain correlation.

FIG. 2A illustrates an exemplary time correlation between inputted by the user subjective pain measurements and the sensor data obtained from the sensors of the wearable device. As noted above, the user may provide subjective pain measurements through the microphone described above.

With reference to FIG. 2A, the Y-axis represents user-inputted subjective pain intensity using black dots correlated with sensor data being illustrated using white squares. The sensor data can measure, e.g., one or multiple physiological parameters of the user, such as blood pressure, pulse or temperature of the user at the time the user is experiencing pain. By combining both the subject pain intensity along with the sensor data, a correlation between the pain intensity and sensor data may be generated for each occurrence of experienced pain. This correlation may be an exemplary training that can be used to match the user-inputted subjective level of pain and the corresponding obtained sensor data.

In another embodiment, sensor data related to movement data may be correlated with user-inputted subjective pain to obtain information about what types of movements may cause the user pain for a scenario where the user is recovering from, for example, a broken or twisted arm. Movement of the arm in certain directions may cause the user pain. Sensor data may be able to capture the user arm movements and match the sensor data with the subjective levels of pain provided by the user. Use of this matching may be helpful to monitor over time the movement by the user and corresponding pain experienced to determine what types of movements cause the user pain and if the user is recovering properly. The matching may also be helpful in evaluating if the pain experienced by the user may be induced by another reason.

FIGS. 2B and 2C both illustrate exemplary embodiments where the correlation can be performed for a specific period of time, e.g., to show whether an injury is healing or non-healing. With reference to FIG. 2B, an example data set of trained sensor data pain levels are plotted on the graphical display with respect to corresponding pain intervals. For example, the data in FIG. 2B illustrates that the user experiences a high level of pain (e.g., level 9) at intervals of five to twenty five seconds on the first day. Over time, however, the pain intensity decreases. Further, the corresponding interval for each occurrence of pain also decreases. In this way, plot B may illustrate an example situation where the user may be properly healing over time. A healing curve can be provided to generalize how a proper recovery process may look like associated with a particular pain-related injury.

FIG. 2C, on the other hand, may illustrate a situation where healing may not be proper/successful. For example, as shown in FIG. 2C, even though the pain may be slightly subsiding over time, the frequency and duration of pain can increase. A corresponding non-healing curve can also be obtained to generalize how a non-healing recovery process may look like for a particular pain-related injury.

FIG. 2D illustrates an exemplary correlation of the intensity of the pain with different combination of the measurements taken over a period of time. In this example, different combinations of the sensor data illustrated as a sign “x” and taken with different frequency are correlated with different levels of pain experienced by the user.

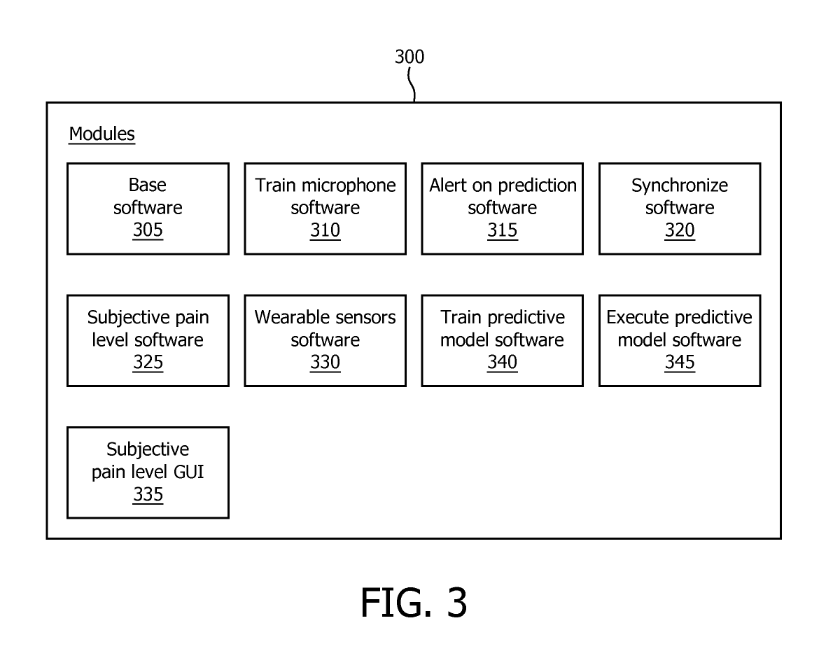

FIG. 3 illustrates exemplary pain management wearable base software. In particular, the figure shows the various modules that may be included in the pain management wearable base software 300. It should be noted that other modules may also be included not illustrated in the figure. In any case, these additional modules may still be useful in carrying out the functionality of the pain management wearable device.

The pain management wearable base software 300 may include, for example, base software 305, a train microphone software 310, alert on prediction software 315, synchronize software 320, subjective pain level software 325, wearable sensors software 330 and subjective pain level GUI 335. The pain management wearable base software 300 can also include software 340 for training the predictive model and software 345 for executing the predicting model to predict the intensity level of the pain of the user It should be noted that the pain management wearable base software 300 of FIG. 3 may be the same pain management wearable base software 161 illustrated in FIG. 1.

The base software 305 included in the pain management wearable base software 300 may be a module within the pain management wearable base software 300 that is responsible for the management and operation of the pain management wearable device. As described above, the base software 305 may instruct the pain management wearable device to collect sensor data used to quantify and record user bodily pain. The base software 305 can manage and run all the other pieces of software that is included in the pain manage wearable base software 300. Further details are provided below in FIG. 5.

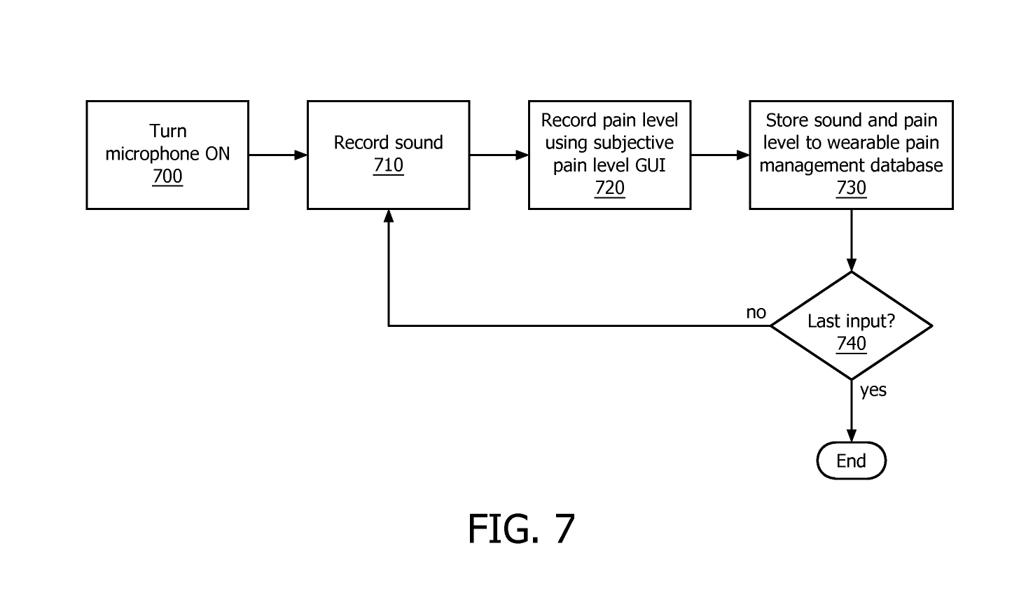

The train microphone software 310 may be used by the pain management wearable device to match user inputted subjective pain measurements with sensor data obtained by the pain management wearable device. As described above, the pain management wearable device may be capable of correlating data between the subjective pain measurements and sensor data for each occurrence of pain to determine a relationship that can be used to quantify, for example, a verbal signal spoken into the microphone (e.g., a user-inputted subjective pain measurement) with sensor data measuring the same occurrence of pain. The training may also be capable of assigning different verbal signals with different pain intensities. For example, the verbal signal representing a groan can be assigned to the intensity level of pain equal seven, whereas the verbal signal representing a grunt can be assigned to the intercity level of pain equal three. Further details are provided below in FIG. 7.

The alert on prediction software 315 may be used to notify the user in situations where an occurrence of pain may be predicted. As indicated above in FIG. 2D, it may be possible for the pain management wearable device to evaluate past data of user experienced pain to predict future occurrences of pain and corresponding intensity. The alert on prediction software 315 may be instructed to provide an alert to the user (e.g., vibration using the vibrator) when a predicted occurrence of pain is above a pre-defined threshold or violates a rule.

The synchronize software 320 may be used to synchronize the information stored in the memory of the pain management wearable device and the pain management receivers electronic device. The synchronization may be desired in situations where the pain management receivers electronic device is implemented in assisting the functionality of the pain management wearable device.

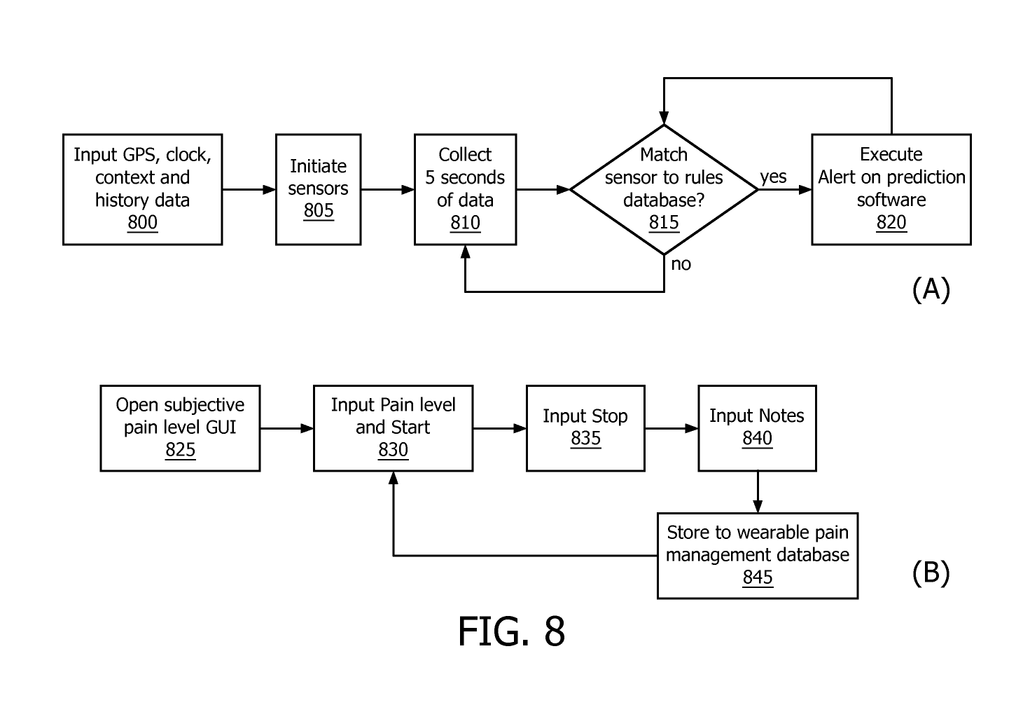

The subjective pain level software 325 may be used, in combination with the subjective pain level GUI 335, to obtain user inputted subjective pain levels for a particular occurrence of pain. The subjective pain level software 325 may extract the user inputs and store the user inputs into memory to be used for pain predictions. Further details are provided below in FIG. 8B.

The wearable sensors software 330 may be used to instruct one or more sensors to obtain sensor data. The sensor data may be directed at biometric parameters (e.g., blood pressure, pulse, temperature) of the user corresponding to a currently experienced occurrence of pain. The wearable sensors software 330 may then store the sensor data into memory to be used later for pain predictions. Further details are provided below in FIG. 8A and FIG. 10A.

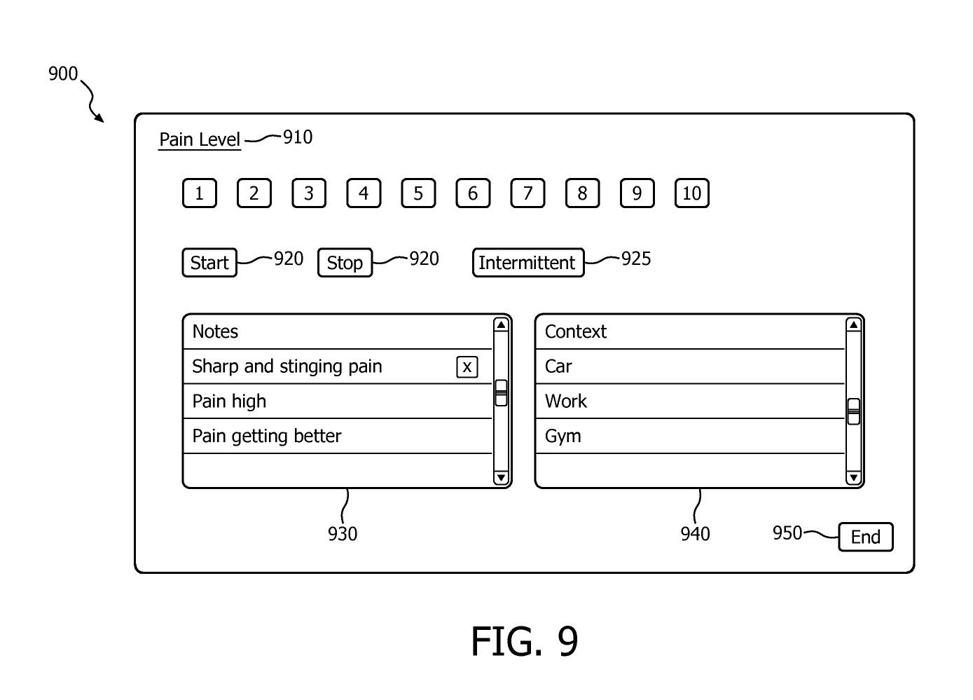

The subjective pain level GUI 335 may also be used to obtain user-inputted subjective pain measurements. In particular, the user may provide the subjective pain measurements through the use of the pain management wearable device (e.g., display and input elements). Further details are provided below with FIG. 9.

The “train predictive model software” 340 is used to determine the predictive model that is used for predicting the intensity level of the user. In one embodiment, the predictive model is trained in advance by correlating the intensity levels of occurrences of pain experienced by the user with physiological and activity data of the user. Additionally or alternatively, one embodiment updates the predictive model in response to receiving the time and the intensity level of the occurrence of pain experienced by the user.

The “execute predictive model software” 345 is used to predict the predicting the intensity level of the pain based on the predictive model and the values of the physiological parameter and the activity of the user. In some embodiments, the predictive model estimates an intensity level of pain as a function of at least one physiological parameter of a user of the wearable device and at least one activity of the user. The software 345 receives the physiological parameter and the activity of the user obtained using various sensors of the wearable device and predicts the intensity level of the pain based on the predictive model and the values of the physiological parameter and the activity of the user.

FIG. 4 illustrates exemplary wearable pain management GUI. As indicated above, the wearable pain management GUI 400 may be used to manage and customize operation of the pain management wearable device.

The wearable pain management GUI 400 may have a feature that allows the user to call up their profile via interaction with a profile button 405. The user profile may include information such as the user name, age, weight, and user identification. The profile may also allow the user to view reports and results of the pain they experienced or are experiencing. These reports may have been generated by the pain management wearable device in the past and stored in memory.

The wearable pain management GUI 400 may also include a profile 405 for one or more sensors available on the pain management wearable device. The wearable pain management GUI 400 may allow the user to turn on or off one or more of the sensors. For example, the user may turn on or off use of the accelerometer, blood pressure sensor, temperature sensor or GPS element.

It should be noted that more and different type of sensors known in the art can be included in other embodiments that are not currently illustrated in FIG. 4. In such embodiments, the user may be able to add additional sensors.

The wearable pain management GUI 400 may also include other options with respect to the microphone associated with the pain management wearable device 415. For example, the user may be allowed to turn the microphone on or off, may initiate training of the microphone to associate auditory noises (e.g., cries, groans, words) with corresponding sensor data for pain, or may allow the pain management wearable device to request subjective user input for currently experienced pain intensity. The wearable pain management GUI 400 may also allow the user to enable alerts to be provided to the user if prediction for pain reaches a particular pain threshold or violates some rule of concern.

The wearable pain management GUI 400 may also allow the user to use additional data when calculating a pain prediction. Although not necessary for pain predictions, the user may enable use of context data and long-term history data 420. The use of context data may provide another factor that can be used to inform whether a user location influences occurrences of pain. This type of data may not be available if, for example, the GPS element is not used. Similarly, long-term history data may be usable in providing an overview of any patterns for occurrences of pain. The use of long-term history data may be disabled, for example, if the user does not wish to have that data considered.

The wearable pain management GUI 400 may also include an option to synchronize 425 data between the pain management wearable device and the pain management receivers electronic device. This option may be chosen, for example, if the user would like to utilize the pain management receivers electronic device to assist in the functionality of the pain management wearable device.

FIG. 5A illustrates an exemplary method for the base software of the pain management wearable device. As described above, the base software may be responsible for the management and operation of the pain management wearable device through the managing and running of the various modules and software included in the pain management wearable device.

In step 500, the base software initiates the wearable pain management GUI. This may allow the user to manage and customize the operation of the pain management wearable device as described above.

Once the wearable pain management GUI has been filled out, the microphone can be used to train 510 the acoustic model for recognizing different level of pains indicated by user utterances. During the training 510, the training microphone software is initialized. As described above, the training microphone software may be used to correlate user-input subjective pain measurements obtained from the microphone (e.g., verbal signals) with sensor data that measures, for example, user biometrics and other parameters that may be associated with the same occurrence of pain.

During the training, acoustic events are segmented from the digitized microphone signal by comparing spectral and amplitude signatures of the signals to a model of the background sound level and noises. The audio signal data from the segmented events is then converted to numeric features suitable for the automatic classification of the sound events. Typical examples of such representations are numeric features corresponding to short-term amplitude level, pitch, spectrum centroid, and tonality features, Mel-frequency cepstrum coefficient features typically used in automatic speech recognitions, or automatically generated feature representations for example in deep neural networks. The events are then classified to a number classes corresponding to a set of predefined pain-related vocalizations. The classified events can be displayed to a user (e.g., graphically, textually, etc.) with their timestamps to indicate chronology. A user can review the acoustic events, select one or more events, and update/change the classification.

In step 520, the base software can initiate the subjective pain level software. The subjective pain level software, in conjunction with the subjective pain level GUI, can obtain user input of a subjective pain level using, for example, the touch display and/or input elements of the pain management wearable device. These additional user inputted subjective pain level measurements can also be used and correlated with the information used during the training of the microphone in step 510. For example, the subjective pain level measurements can be correlated with different classes of the auditory inputs. The correlations may also be computed between the computational features of the classifier and the subjective pain level measurements. In step 530, the base software may execute the wearable sensor software. This may provide instructions to the various sensors associated with the pain management wearable device to obtain sensor data corresponding to occurrences of pain that the user experiences. The sensors may constantly poll for available sensor data or may be triggered to obtain data based on a condition (e.g., receipt of a user input from the microphone or subjective pain level GUI).

The sensors may continually poll for sensor data until either the sensors are instructed to stop (e.g., by the user) or after a set time limit. In situations where sensors obtain sensor data that leads to a calculated pain prediction that exceeds a pre-defined threshold value or violates some rule, the base software may execute the alert on prediction software in step 540 to notify the user about a particular pain prediction. In this way, the user can be given notice in order to, for example, undergo preventative measures aimed at mitigating the experienced future pain.

FIG. 5B illustrates a block diagram of a system for recognizing different level of pains from auditory inputs. For example, the user's acoustic inputs are received through a microphone 501, and transmitted to a server 503, such as an ASP, via a network 502. The server 503 may include a database, memory or other storage device 504 that can retain previous voice samples of the user and/or data related to the user.

The pre-processing module 505 can evaluate the condition of the signal and perform signal conditioning. The signal conditioning can include, but is not limited to, removing contaminated segments and/or filtering the signal. The pre-processing module 505 can reduce noise in the signal. In one embodiment, the pre-processing module 505 can be used to select the auditory inputs for further analysis. In one embodiment, after performing the pre-processing, an auditory-based or other non-linear transformation, such as a logarithmic transformation, can be applied as a front end for signal processing before the signal is analyzed.

The user's acoustic inputs are analyzed according to predetermined metrics (acoustic measures) in a speech metrics module 506. For example, acoustic analysis can be performed to quantify metrics including, but not limited to fundamental frequency characteristics, intensity, articulatory characteristics, speech/voice quality, prosodic characteristics, and speaking rate. For language analysis, the user's language is analyzed for language patterns in a language marker module 515. The language marker module 515 can include an automatic speech recognition (ASR) module. After performing the speech and/or language analysis, modeling and coding can be performed by the coding module 511 via statistical approaches, machine learning, pattern recognition, or other algorithms to correlate the acoustics inputs of the user with different levels of the pain.

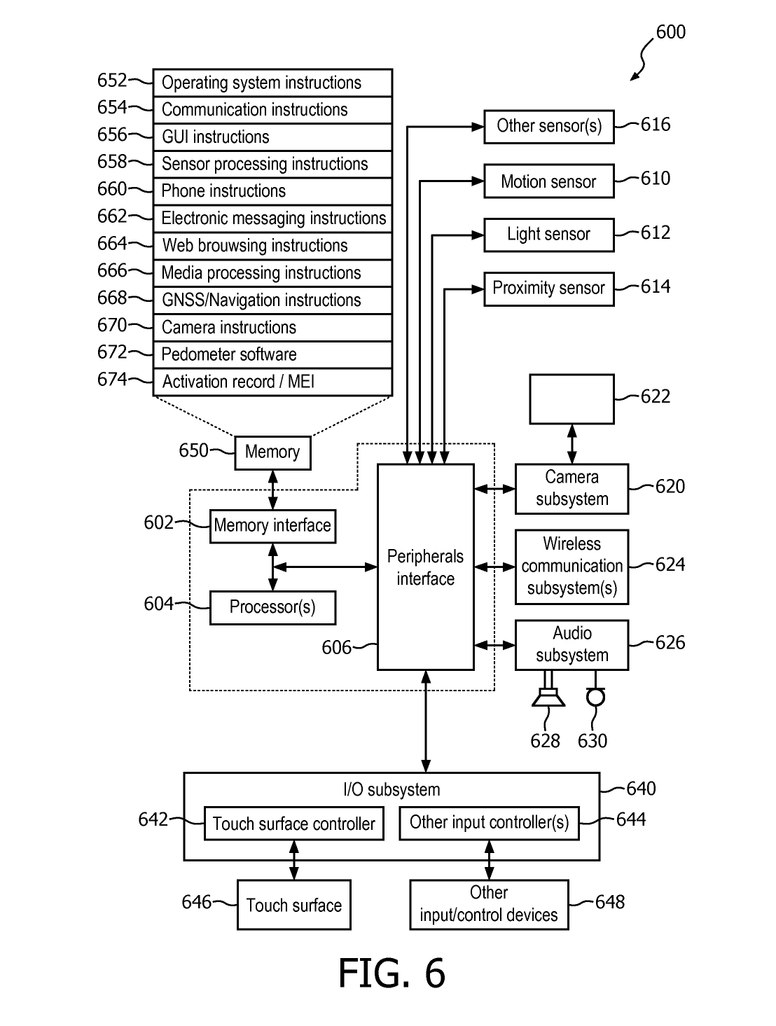

After the information from the speech and/or language analysis is obtained, comparators 512 can be used to reach a correlation decision. For example, in one embodiment, the acoustic inputs are compared to a normative data set (norm-based test), such as the baseline acoustic measures stored in a memory or other storage device 513 connected with the comparator. FIG. 6 illustrates an exemplary computing device architecture that may be utilized to implement the various features and processes described herein. For example, the computing device architecture 600 could be implemented in a pedometer. Architecture 600 as illustrated in FIG. 6 includes memory interface 602, processors 604, and peripheral interface 606. Memory interface 602, processors 604 and peripherals interface 606 can be separate components or can be integrated as a part of one or more integrated circuits. The various components can be coupled by one or more communication buses or signal lines.

Processors 604 as illustrated in FIG. 6 is meant to be inclusive of data processors, image processors, central processing unit, or any variety of multi-core processing devices. Any variety of sensors, external devices, and external subsystems can be coupled to peripherals interface 606 to facilitate any number of functionalities within the architecture 600 of the exemplar mobile device. For example, motion sensor 610, light sensor 612, and proximity sensor 614 can be coupled to peripherals interface 606 to facilitate orientation, lighting, and proximity functions of the mobile device. For example, light sensor 612 could be utilized to facilitate adjusting the brightness of touch surface 646. Motion sensor 610, which could be exemplified in the context of an accelerometer or gyroscope, could be utilized to detect movement and orientation of the mobile device. Display objects or media could then be presented according to a detected orientation (e.g., portrait or landscape).

Other sensors could be coupled to peripherals interface 606, such as a temperature sensor, a biometric sensor, or other sensing device to facilitate corresponding functionalities. Location processor 615 (e.g., a global positioning transceiver) can be coupled to peripherals interface 606 to allow for generation of geo-location data thereby facilitating geo-positioning. An electronic magnetometer 616 such as an integrated circuit chip could in turn be connected to peripherals interface 606 to provide data related to the direction of true magnetic North whereby the mobile device could enjoy compass or directional functionality. Camera subsystem 620 and an optical sensor 622 such as a charged coupled device (CCD) or a complementary metal-oxide semiconductor (CMOS) optical sensor can facilitate camera functions such as recording photographs and video clips.

Communication functionality can be facilitated through one or more communication subsystems 624, which may include one or more wireless communication subsystems. Wireless communication subsystems 624 can include 802.x or Bluetooth transceivers as well as optical transceivers such as infrared. Wired communication system can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 624 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.x communication networks, code division multiple access (CDMA) networks, or Bluetooth networks. Communication subsystem 624 may include hosting protocols such that the device may be configured as a base station for other wireless devices. Communication subsystems can also allow the device to synchronize with a host device using one or more protocols such as TCP/IP, HTTP, or UDP.

Audio subsystem 626 can be coupled to a speaker 628 and one or more microphones 630 to facilitate voice-enabled functions. These functions might include voice recognition, voice replication, or digital recording. Audio subsystem 626 in conjunction may also encompass traditional telephony functions.

I/O subsystem 640 may include touch controller 642 and/or other input controller(s) 644. Touch controller 642 can be coupled to a touch surface 646. Touch surface 646 and touch controller 642 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 646 may likewise be utilized. In one implementation, touch surface 646 can display virtual or soft buttons and a virtual keyboard, which can be used as an input/output device by the user.

Other input controllers 644 can be coupled to other input/control devices 648 such as one or more buttons, rocker switches, thumb-wheels, infrared ports, USB ports, and/or a pointer device such as a stylus. The one or more buttons (not shown) can include an up/down button for volume control of speaker 628 and/or microphone 630. In some implementations, device 600 can include the functionality of an audio and/or video playback or recording device and may include a pin connector for tethering to other devices.

Memory interface 602 can be coupled to memory 650. Memory 650 can include high-speed random access memory or non-volatile memory such as magnetic disk storage devices, optical storage devices, or flash memory. Memory 650 can store operating system 652, such as Darwin, RTXC, LINUX, UNIX, OS X, ANDROID, WINDOWS, or an embedded operating system such as VxWorks. Operating system 652 may include instructions for handling basic system services and for performing hardware dependent tasks. In some implementations, operating system 652 can include a kernel.

Memory 650 may also store communication instructions 654 to facilitate communicating with other mobile computing devices or servers. Communication instructions 654 can also be used to select an operational mode or communication medium for use by the device based on a geographic location, which could be obtained by the GPS/Navigation instructions 668. Memory 650 may include graphical user interface instructions 656 to facilitate graphic user interface processing such as the generation of an interface; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GPS/Navigation instructions 668 to facilitate GPS and navigation-related processes, camera instructions 670 to facilitate camera-related processes and functions; and instructions 672 for any other application that may be operating on or in conjunction with the mobile computing device. Memory 650 may also store other software instructions for facilitating other processes, features and applications, such as applications related to navigation, social networking, location-based services or map displays.

Each of the above identified instructions and applications can correspond to a set of instructions for performing one or more functions described above. These instructions need not be implemented as separate software programs, procedures, or modules. Memory 650 can include additional or fewer instructions. Furthermore, various functions of the mobile device may be implemented in hardware and/or in software, including in one or more signal processing and/or application specific integrated circuits.

Certain features may be implemented in a computer system that includes a back-end component, such as a data server, that includes a middleware component, such as an application server or an Internet server, or that includes a front-end component, such as a client computer having a graphical user interface or an Internet browser, or any combination of the foregoing. The components of the system can be connected by any form or medium of digital data communication such as a communication network. Some examples of communication networks include LAN, WAN and the computers and networks forming the Internet. The computer system can include clients and servers. A client and server are generally remote from each other and typically interact through a network. The relationship of client and server arises by virtue of computer programs running on the respective computers and having a client-server relationship to each other.

One or more features or steps of the disclosed embodiments may be implemented using an API that can define on or more parameters that are passed between a calling application and other software code such as an operating system, library routine, function that provides a service, that provides data, or that performs an operation or a computation. The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer will employ to access functions supporting the API. In some implementations, an API call can report to an application the capabilities of a device running the application, such as input capability, output capability, processing capability, power capability, and communications capability.

FIG. 7 illustrates an exemplary method for training a microphone. As noted above, the microphone can be used to obtain subjective auditory signals from the user (e.g., auditory sounds, words) for quantifying and recording pain intensity. Training can be performed, via the train microphone software, to associate the subjective input with sensor data measured by the pain management wearable device.

In step 700, the training of the microphone may be initiated when the microphone is turned on. The training may occur automatically if the microphone for the pain management wearable device has not yet been trained. Subsequent use of the microphone may still automatically initiate the training. There may be embodiments, however, where training option(s) for the microphone can be selected or enabled once and the settings saved for future use. In such an instance, the microphone may be turned on when the user is currently experiencing pain. At this point, the train microphone software may instruct the microphone to proceed to the next step.

In step 710, the train microphone software can then instruct the microphone to record various inputs (e.g., auditory sound, words) from the user. In particular, the inputs should be indicative of a particular level of pain that the user is currently experiencing. For example, a soft sound can be used to indicate low amounts of pain while a louder grunt could be used for intense pain.

In step 720, subjective inputs obtained by the microphone can then be compared with the inputs obtained from the user through the subjective pain level GUI. As discussed below, the subjective pain level GUI allows the user to quantify a level of pain being experienced. For example, a low amount of pain may be given a value of 1-3 while more intense pain may be given a higher value of 6-9.

In step 730, the various auditory inputs obtained in step 710 and the quantified subjective pain level value obtained in step 720 can be correlated. This correlation can then be stored within the pain management wearable device (e.g., wearable pain management database.

In step 740, the train microphone software may continually correlate auditory inputs and inputs from the subjective pain level GUI so long as the user continues to provide inputs that the train microphone software can use. Once the last input has been received, the train microphone software can terminate.

It should be noted that the training of the microphone can be performed by the user and other medical professionals as well. For example, a doctor may wish to measure and record certain body movements after surgery to determine what body movements may cause pain. The doctor may train the microphone of the pain management wearable device by instructing the user to move in particular manners and recording the user reactions in order to correlate subjective levels of pain from the user with measured pain obtained at the same time from the sensors.

FIG. 8A illustrates an exemplary method for the wearable sensor software. As described above, the wearable sensor software may provide instructions to the various sensors associated with the pain management wearable device to obtain sensor data corresponding to occurrences of pain that the user experiences.

In step 800, the wearable sensor software may take as inputs various data (e.g., GPS, clock, context and long-term history data). These inputs may be later used by the wearable sensor software (see step 820) if situations arise where an alert is necessitated. For example, an alert may be necessary if the sensor data obtained by the wearable sensor software exceeds a pre-defined threshold or violates some rule.

In step 805, the wearable sensor software can then initiate the various sensors. The sensors may be initiated, for example, upon the receipt of user input of subjective pain levels. The user input can signal that sensor data should be taken at the same time so that the sensor data can be used to correlate with the subjective pain level.

In step 810, the wearable sensor software can instruct the various sensors to collect data at pre-defined intervals (e.g., every 5 seconds). It should be noted that the intervals may be customized based on the preferences of the user.

In step 815, the wearable sensor software then evaluates the predicted intensity level of pain with rules stored in a memory of the wearable device and executing one or more actions based on the evaluation of the predicted intensity level of pain with the rules. For example, if any rules are triggered through the comparison, an alert can be sent to the user through the use of the alert on prediction software (step 820). The alert may be provided to the user in various ways, for example, producing a vibration using the vibrator or displaying a message on the display of the pain management wearable device.

If no rules were violated, however, the wearable sensor software can instruct the pain management wearable device to continue polling again for more sensor data. As noted above in step 810, the sensors may be instructed to continually poll for data at regular intervals (e.g., 5 seconds). This loop produced between steps 810 and 815 may repeat until an alert is triggered or the wearable sensor software is provided instructions to terminate (e.g., the user shuts down the pain management wearable device).

FIG. 8B illustrates an exemplary method for the subjective pain level software. As indicated above, the subjective pain level software, in conjunction with the subject pain level GUI, can obtain user input quantifying an experienced level of pain (pain intensity) that the user is currently experiencing.

In step 825, the subjective pain level software may initiate the subjective pain level GUI. The subjective pain level GUI allows the user to provide a numerical value, among other information, related to an experienced level of pain. Further details are provided blow in FIG. 9. The input can be, for example, a subjective rating of the pain being experienced by the user on a scale from one to ten where one represents very little pain and ten representing extreme pain.

In step 830, the subjective pain level software can then input the values provided by the user through the subjective pain level GUI. These values may continually be inputted from the subjective pain level GUI. For example, the user may be instructed to provide input at regular intervals by the pain management wearable device in order to gauge how the pain peaks and/or subsides over time.

In step 835, the subjective pain level software may be instructed to top taking further inputs. This might correlate with a situation whereby the pain that the user was experience had completely subsided.

In step 840, the subjective pain level software can also take note of other information related to the level of pain being provided. For example, the subjective pain level software may keep track of a time duration of the pain being experienced by the user. The subjective pain level software may also allow the user to make any subjective notes regarding the experienced pain for future reference (e.g., describing how the pain felt). The user may also be allowed to provide a context of where the pain was experienced.

In step 845, the subjective pain level software can then take the various subjective pain level inputs provided by the user and store them into the wearable pain management database.

FIG. 9 illustrates an exemplary subjective pain level GUI. As described above, the subjective pain level GUI 900 facilitates the user in providing a subjective rating of an experienced pain intensity 910. For example, the pain intensity can be ranked on a scale from one to ten, whereby one references slight pain while ten references intense pain. In some embodiments the estimate of the subjective pain is given by a second person which may be a caregiver of the subject.

The subjective pain level GUI may also include a start and stop button 920 to facilitate in the measurement of the duration of the occurrence of pain. An intermittent button can be used to signal that the pain is sporadic and may not be easily quantified using the start and stop button.

Additional notes 930, furthermore, can be associated with each particular occurrence of pain. The notes could be selected from a list of already existing notes in a menu or provided as an input from the user. These notes can be used to describe the type of pain or what the user is thinking about the pain at the time.

The user may also provide context 940 associated with the pain being experienced. In situations where the GPS is not used, the user may provide location information that can be used to correlate, for example, whether a location impacts the pain being experienced by the user. For example, if the user continually experiences pain while at work, this may be indicative of poor posture (e.g., sitting/slouching).

The user may be instructed to provide inputs at regular intervals using the subjective pain level GUI. The user may also provide inputs whenever the user feels like there has been a change, for example, in the pain intensity. These multiple user inputs can be used to monitor a progression of the pain as time goes on. Once the pain has subsided or the user no longer desires to provide further inputs using the subjective pain level GUI, the user can close the GUI and subsequently terminate the subjective pain level software by interacting with the end button 950.

FIG. 10A illustrates an exemplary situation for the wearable sensors software. As referenced above in FIG. 8A, the wearable sensor software can instruct the sensors of the pain management wearable device to obtain 1010 sensor data relating to an experienced level of pain. The data can be collected 1020 at regular intervals (e.g., every five seconds). Using the obtained sensor data, the wearable sensors software compares 1030 the obtained sensor data with user inputted subjective pain levels where such subjective pain levels may have been provided via the subjective pain level GUI.

In the embodiment of FIG. 10A, a rule may exist whereby an alert would be provided to the user if an experienced pain is detected at a rating of 6 or higher. So for each set of sensor data obtained at an interval, a matching 1040 can be performed between the sensor data (and corresponding subjective pain level) and the rule (further illustrated in FIG. 10B). Based on any existing matches between the rules and the obtained sensor data, the wearable sensor software executes 1050 actions and provides comments to the user based on the instructions associated with the matched rule.

FIG. 10B illustrates an exemplary rules database 1060. As indicated above, the rule database may include a variety of conditions that may trigger an alert (e.g., pain intensity threshold). Based on a match between one or more of the rules and the obtained sensor data, a corresponding action may be executed and/or a corresponding comment may be provided to the user.

As an example, as shown in FIG. 10B, a rule may set a subjective threshold pain level for the user at six. For measurements between zero and five, there may be no action. If measurements are obtained going from five to six, a single vibration may be provided to the user with a corresponding comment that a projection or pain may occur. Further escalation of the pain past the subjective threshold may provide further vibrations with additional comments indicating the severity of the pain (e.g., pain high projection, pain at height).

Also shown in the figure are corresponding example actions and comments for situations where the subjective threshold pain level reduces over time. It should be noted that the rules database may include different thresholds, values and determinations for triggering corresponding actions and comments to be performed for the user.

FIG. 11 illustrates an exemplary receiver GUI 1110. As noted above, the receiver GUI is stored in memory found on the pain management receiver electronic device. The receiver GUI may facilitate in the synchronization of data between the pain management wearable device with the receiver electronic device. The user can enable synchronization to occur between the pain management wearable device and the pain management receiver electronic device. The user may also request reports summarizing information stored in the pain management receiver electronic device by interacting with the “show reports” button. The reports may include information relating to a summary of pain intensity and frequency of pain occurrences for the user to view on the display of the pain management receiver electronic device.

FIG. 12A illustrates an exemplary long-term history database. As noted above, data relating to occurrences of pain experienced by the user may be stored in a database for long-term reference. The data may include user inputted subjective pain level along with information identifying the duration of the pain or when the pain may generally be experienced by the user. The long-term history database may be used by the pain management wearable device in providing pain predictions that look at existing long-term patterns and not merely on what is currently being measured. The long-term history database may also be used to evaluate whether the experiences in pain correlate to recovery or a worsening condition.

FIG. 12B illustrates an exemplary context database. As noted above, context data may be helpful in evaluating conditions that may affect occurrences of pain. The data related to the location of the user can be obtained, for example from the subjective pain level GUI or from the GPS. In any case, the current location of the user can be stored alongside the corresponding user inputted subjective pain levels. In this way, the pain management wearable device may be capable of using the user location as a factor in pain prediction.

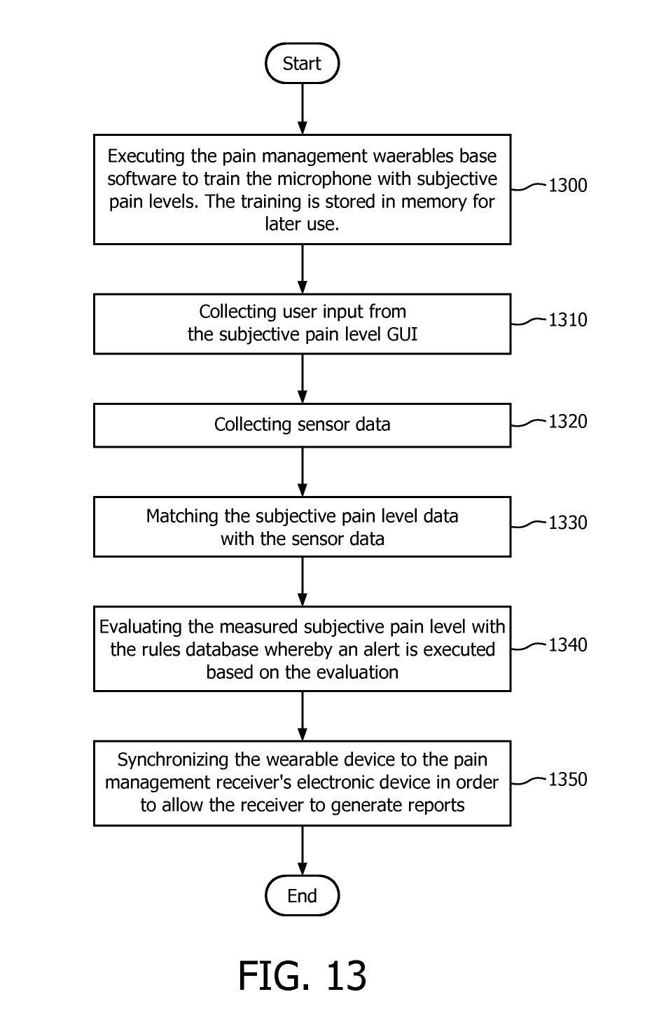

FIG. 13 illustrates an exemplary overall method for pain prediction. In step 1300, the method executes the base software of the pain management wearable device in order to train the microphone. The microphone is used as one way to obtain subjective pain level input from the user. The training compares, for example, the auditory signal (e.g., words, sounds) with other information (e.g., sensor data and input obtained from the subjective pain level GUI) to map a particular signal with a pain intensity level. This training, once completed, can be stored in memory for future use.

In step 1310, the method can then obtain user input regarding a particular experience of pain through the use of the subjective pain level GUI. The subjective pain level GUI provides another way, aside from the microphone, for the user to provide data (e.g., a rating) about experienced pain intensity. The user may also provide notes and location-based data about the pain.

In step 1320, the method can then obtain sensor data from the sensors associated with the pain management wearable device. The sensor data can measure biometric parameters (e.g., blood pressure, temperature, pulse) during experiences with pain.

In step 1330, the user inputs and the sensor data can be matched to see if there are any correlations between the two sets of data. For example, there may be situations where the user's pulse increases as the pain becomes more intense. The sensor data can pick up the user biometric data and the user input can indicate that the pain being experienced is intense. This step can evaluate the two sets of data to see if there is any sort of correlation.

In step 1340, an evaluation of the sensor data and user inputted data can be performed against rules stored in the rules database. The rules may be indicative of situations that the user should be notified about, for example, situations where predicted pain exceeds a pre-defined threshold. If the rules are not violated or there are no matching rules, the method may not provide any notification to the user. If the rules are violated, however, the user may be notified along being provided information about the alert.

In step 1350, synchronization between the pain management wearable device and the pain management receivers electronic device can be performed. Such a synchronization may be desired in order to allow the pain management receivers electronic device to provide additional functionalities (e.g., generating reports) that the pain management wearable device may not be capable of performing.

Some embodiments are based on recognition that the same pain symptoms experienced by a user can be caused by different combinations of different reasons. For example, the back pain can be caused by stress problems, problems with the spine or can just be a result of sleeping on an old mattress or sitting in an uncomfortable position. To that end, some embodiments are based on realization that the pain experienced by the user needs to be determined not only based on the physiological parameters, but also based on other activities of the user.

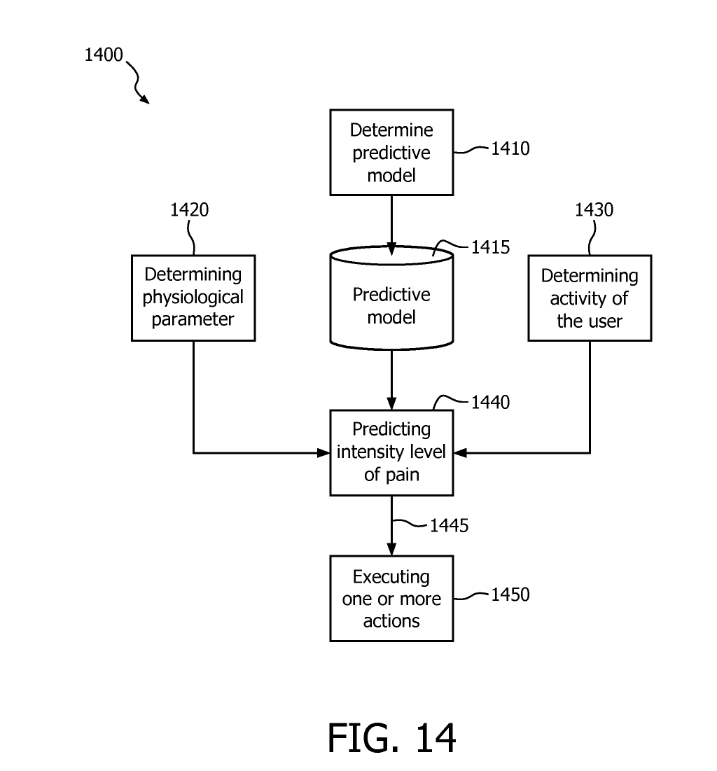

FIG. 14 shows a block diagram of a method 1400 for providing the pain management using a wearable device according to one embodiment. The method can be implemented using a processor of a wearable device. The method determines 1410 a predictive model 1415 estimating an intensity level of pain as a function of at least one physiological parameter of a user of the wearable device and at least one activity of the user. The predictive model 1415 can be determined in advance and stored in the memory of the wearable device. Additionally or alternatively, the predictive model can be updated in response to receiving the time and the intensity level of the occurrence of pain experienced by the user. In various embodiments, the activity of the user includes one or combination of a type of the activity, a level of the activity, a location of the activity, and a duration of the activity. This information individually or collectively provides more details about the activity of the user.

The method 1410 concurrently determines 1420 physiological parameter and determines 1430 the activity of the user. For example, the method concurrently determines measurements of one or more physiological sensors of the wearable device and one or more activity sensors of the wearable device to produce 1420 and 1430 values of the physiological parameter and the activity of the user. As used herein, determining concurrently means determining simultaneously at the same time or sequentially within a brief period of time, e.g., within one minute or a period of time governed by computational capability of the processor of the wearable device.

The method 1400 predicts 1440 the intensity level of the pain 1445 of the user based on the predictive model and the values of the physiological parameter and the activity of the user. Next, the method executes 1450 one or more actions based on the predicted intensity level of pain. For example, the action executed based on the evaluation can notify the user of possibility to have the pain and/or about the cause of the pain by displaying a message on the wearable device.

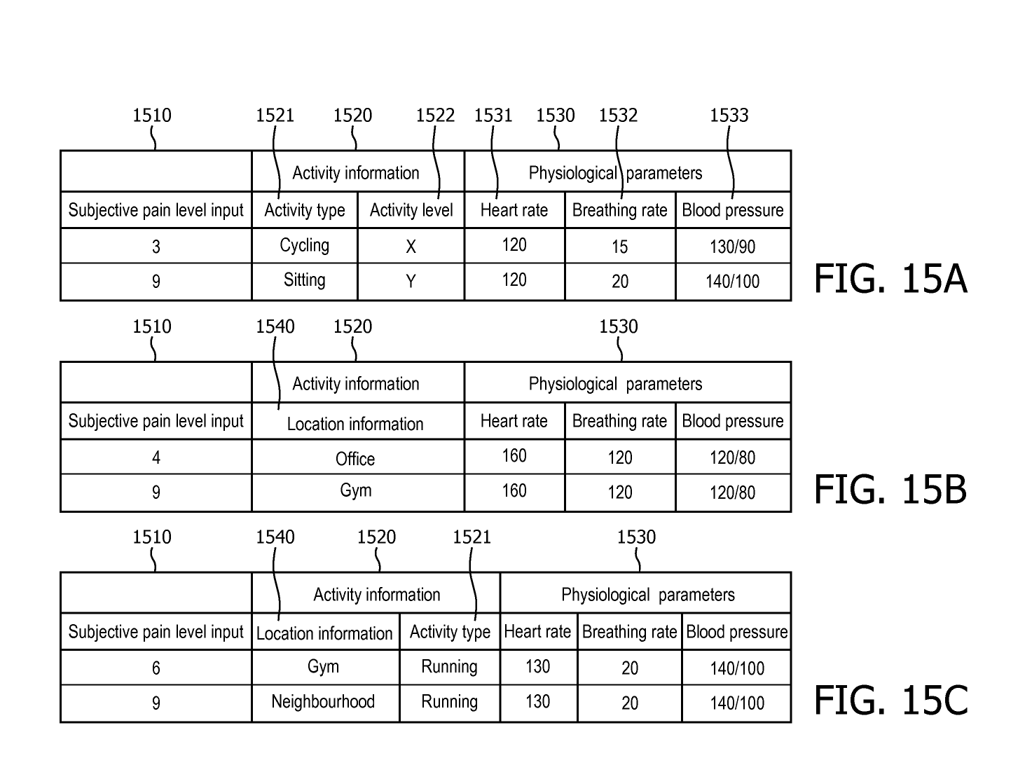

FIG. 15A shows an exemplary table that includes the combination of physiological information 1530, subjective pain level input 1510 and activity information 1520 according to some embodiments. The activity information 1520 further includes an activity type 1521 and an activity level 1522. The table of FIG. 15A emphasizes on the fact that activity information along with physiological information can be used for prediction of pain. For instance, as depicted in the table, the subjective pain level input 1510 indicates that for the same heart rate 1531 and similar breathing rates 1532 and blood pressure 1533; the subjective level inputs 1510 can vary significantly. To that end, some embodiments consider the physiological parameter information in conjunction with the activity information. For instance, only after an activity level has a reached a particular activity threshold, the pain starts to manifest. For instance, sitting for minimum movements for longer duration of time may manifest stronger pain levels than cycling with an activity level that is higher when compared with sitting.

Such information points are collected in the training phase for determining the predictive model that is further used in prediction when the user starts using the device full time/regularly. It can be appreciated by a person skilled in the art that if only physiological information was considered then the prediction could have led to a false positive in which case, the system based on the physiological parameter alone would have predicted that a pain might arise. However, further considering activity information, the system can now discern and better predict the onset of pain based on activity that user is performing. Various predictive models can be used to model these parameters. Few examples include linear regression, neural network, Bayesian network, support vector machine, and the like.

FIG. 15B shows an exemplary table that includes the combination of physiological information, subjective pain level input and location information that forms the predictive model according to another embodiment. The table of FIG. 15B emphasizes on the fact that the location information along with physiological information can be used for prediction of pain. For instance, as depicted in the table of FIG. 15B, for the same heart rate (and same breathing rate and blood pressure; the subjective level input 1510 can vary significantly. Thus, some embodiments consider physiological parameter information 1530 in conjunction with location information 1540. Such, information points are collected in the training phase and are further used in the prediction when the user starts using the device full time/regularly. It can be appreciated by a person skilled in the art that if only physiological information was considered then the prediction could have led to a false positive in which case, the system based on the physiological parameter alone would have predicted that a pain might arise. However, further considering further input as location, the system can now discern and better predict the onset of pain based on location that user is in. Various predictive models can be used to model these parameters. Few examples include linear regression, neural network, Bayesian network, support vector machine, and the like.

FIG. 15C shows an exemplary table that includes the combination of physiological information 1530, subjective pain level input 1510, location information 1540, and activity type 1521 that forms the predictive model according to another embodiment. The table of FIG. 15C emphasizes on the fact that different combinations of the location information and the activity information along with physiological information can be used for prediction of pain. For instance, for the same types of the activity 1521 and the same physiological parameters (1530, the pain levels 1510 can be different based on the location 1540. For instance, when the user is running on gym equipment, such as treadmill, the user can experience less pain when compared with running on the road in the neighborhood. Such, information points are collected in the training phase and are further used in the prediction when the user starts using the device full time/regularly. Various predictive models can be used to model these parameters. Few examples include linear regression, neural network, Bayesian network, support vector machine, and the like.

While not shown, in various embodiments, the records of FIG. 15A, 15B, or 15C may be timestamped in accordance with when the physiological parameters or pain level input, or other values were obtained. Such information may aid in training a predictive model to predict future pain based on present inputs. For example, in the example of FIG. 15A, the predictive model may take the cycling activity as an indicator of the future pain level of “9” rather than the current pain level of “3” when the training set is provided with such a time component. To enable such inferences by the predictive model, additional or alternative training records may be created by combining timestamped records. For example, each capture of activity and physiological parameters may be placed in new record with subjective pain level inputs from other records captured at various points in time after the activity and physiological parameters (e.g., 1, 2, 3, 4, 5, and 6 hours afterward).

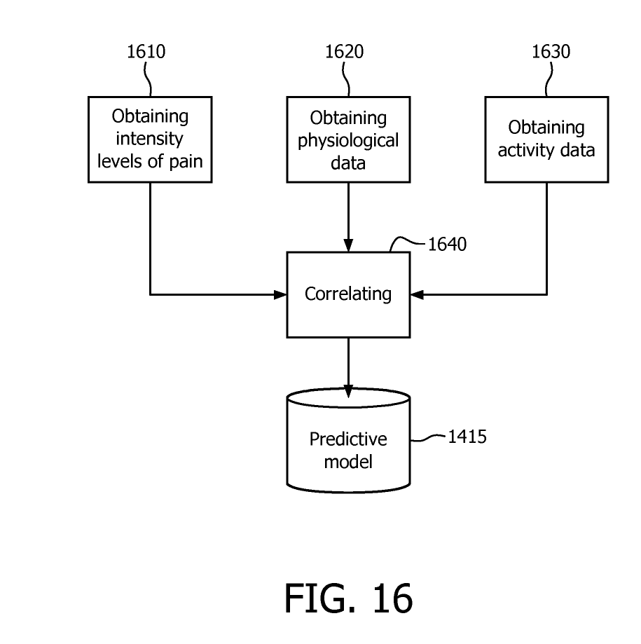

FIG. 16 shows a block diagram of a method for determining the predictive model according to some embodiments. The method obtains 1610 inputs from the user via a user interface, the inputs corresponding to the intensity level of an occurrence of pain experienced by the user. The method also obtains 1620 physiological data from the physiological sensors and obtains 1630 activity data from the activity sensors including one or combination of at least one motion sensor for determining the type of the activity and at least one location sensor for determining the location of the activity. Next, the method correlates 1630 the intensity levels of the occurrence of pain experienced by the user with the obtained physiological data and the activity data to determine the predictive model 1415.

The physiological data and the activity data can be obtained concurrently with the occurrence of pain experienced by the user. In the above disclosed embodiments, the location information can be derived from GPS module 145 of the wearable device 105. In an alternate embodiment, the location information can also be derived from indoor localization, network IP address, etc.

Examples of the sensor for determining the type of the activity include a motion sensor, such as accelerometer, gyroscope, etc. Motion sensor is one of the sensors N included in the device 105. Various solutions are already available to a person skilled in the art to determine activity based on, for instance, accelerometer. In general activity type is recognized by analyzing acceleration signals received from the accelerometer.

In various embodiments various physiological sensors, such as photoplethysmogram (PPG) sensor can be used to monitor heart rate, breathing rate and blood pressure of the user.

Some embodiments obtain the training data for a period of time and determine the predictive model as a regression function correlating different intensity levels of pain with combinations of the physiological data and the activity data. For example, the embodiments can obtain the training data for several weeks or until the required amount of training data needed for determining the predictive model is obtained.

Some embodiments use the training data to determine a regression model which predicts the level of pain Y from a given observations X of the physiological parameters and activity of the user. In one embodiment, the predictive model M is a vector with K coefficients determined using least-squares normal equations for the collected training data. Such a predictive model can be used to predict the pain level as a matrix operation Y=M*X.

Some embodiments are based on recognition that the duration of different activities needs to be used in predicting the intensity level of the pain. In such a manner, the prediction can be performed as a function of time, which allows predicting not only current level of pain, but also a future level of pain and/or the cause of the pain.

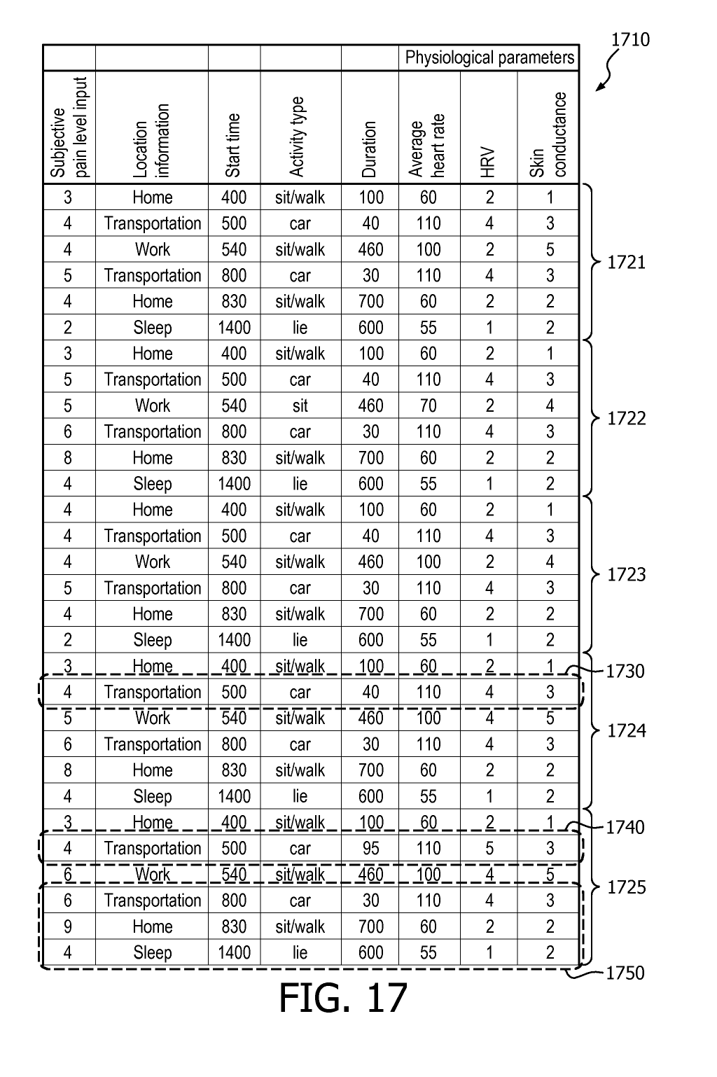

FIG. 17 shows an exemplary table 1710 illustrating different combinations of the physiological parameters and activities of the user for determining the predictive model according to some embodiments. In this example, the activity of the user includes combination of a type of the activity, a location of the activity, and duration of the activity. The data and the pain values are represented as segmented records where the location information, such as “work” or “transportation” are detected based on GPS and time information. The start times and durations of each segment are also indicated in columns. In some embodiments, the type of activity is the most common activity in the segment. Similarly, the physiological parameters such as an average heart rate, heart rate variability (HRV), and skin conductance are mean values in the segment.

In this example, the time period for collecting training data is partitioned into days, such as a day 1721, a day 1722, a day 1723, a day 1724, and a day 1725. For example, the data collected for the day 1721 represent a typical day with no pain. For example, on the day 1722 the user is mostly sitting and not moving much at work which is indicated by the lower average heart rate and lot of sitting in the work segment.

The day 1724 is another case where a stress measure related to heart rate variability (HRV), and skin conductance of the subject are higher than usual in the work segment. This may indicate a stressful work day which in this case leads to an increase the subjective pain in the following segments. In the exemplar day 1725, the user gets stuck in a traffic jam in the first transport segment, had an elevated stress indicator based on HRV and gets the back pain from sitting in the car for a too long time.

Some embodiments are based on recognition that among all factors contributing the level of pain of the users, at different points of time and/or for different circumstances, some factors contribute more to the increase/decrease of the level of pain than the others. For example, the comparison of a data set 1730 with a data set 1740 can indicate that in those circumstances, the level of pain is less sensitive to variations in HRV, skin conductance or hear rate, but more sensitive to duration and the type of the “transportation” activity. Therefore, next time when the extended period of traveling in the car is detected, e.g., e.g., for 60 minutes, the wearable device according to different embodiments can notify the user that the continuation of such an activity can cause the bodily pain. Notably, such a prediction can be determined before the actual occurrence of the pain. In other circumstances, the pain level can be more sensitive to physiological parameters. For example, as can be seen in the data set 1750, the reduction of the heart rate can lead to the reduction of the level of pain. To that end, the wearable device can suggest to the user the activity and/or the medicine that can reduce the heart rate.

Some embodiments determines the correlation between various physiological parameters and activities of the user as a multi-dimensional regression function, wherein a particular dimension of the regression function corresponds to values of a particular physiological parameter or a particular activity of the user.

Some embodiments are based on understanding that it is not always practical to expect a perfect match between the current physiological parameters and activities of the user and the training data. Accordingly, some embodiments use regression analysis as a statistical process for estimating the relationships between the different combinations of the values of the physiological parameters and activities of the user and corresponding values of the level of pain. For example, one embodiment trains a regression function establishing such a relationship. In that embodiment, the regression function is a multi-dimensional function, wherein a particular dimension of the regression function corresponds to values of a particular physiological parameter or a particular activity of the user.

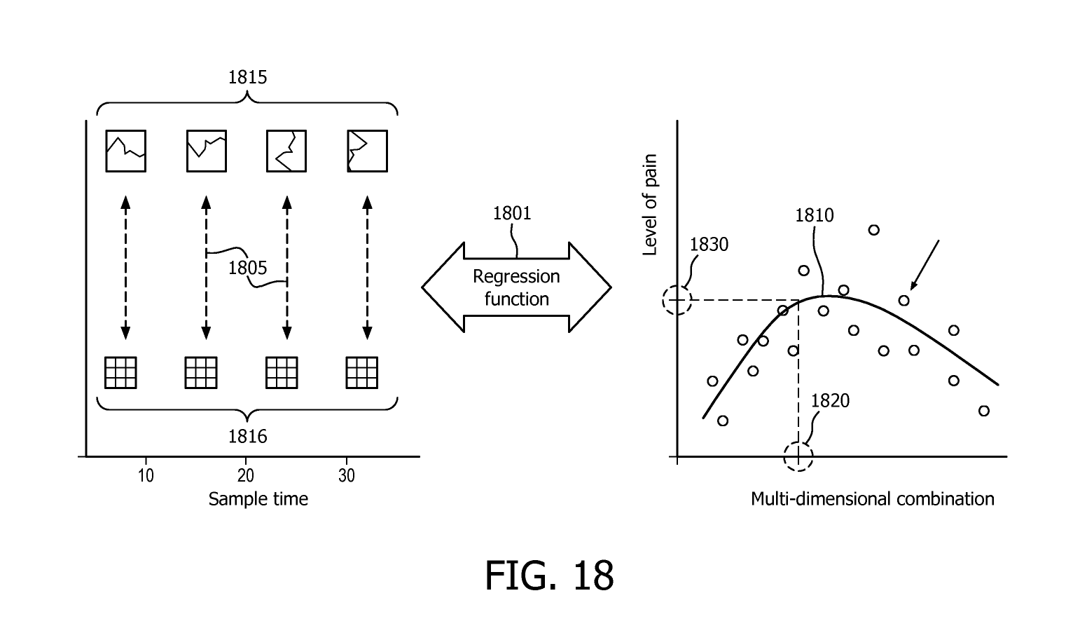

FIG. 18 shows a schematic of training 1801 the regression function 1810 according to one embodiment. The regression function establishes a correspondence 1805 between the different combinations 1816 of the values of the physiological parameters and activities of the user and corresponding values of the level of pain 1815. Knowing the regression function 1810, the particular level of pain 1830 can be determined from the particular observations 1820 of the values of the physiological parameters and activities of the user. The combination of values can be of any dimensions. For example, in the example of the FIG. 17, such a combination can have up to seven dimensions, one for each column of the table 1710 with the exception of the column specifying the level of pain. The regression function 1810 can be any complex function. For example, the regression function can be linear, nonlinear, and nonparametric regression function. In some embodiments, the regression function can be a polynomial function or a spline.

Advantageously, the regression function 1810 allows determining sensitivity of the regression function to the variation of values along different dimensions of the regression function. For example, the sensitivity can be determined by taking partial derivatives of the regression function for different dimensions of the regression function. The values of the partial derivatives at the point corresponding to the current observations 1820 of the values of the physiological parameters and activities of the user indicate the sensitivities of the regression function at that point. In addition, signs of the partial derivatives indicate the direction of the variation, i.e., whether the increase or decrease of the value along one dimension leads to an increase or decrease of the level of pain.

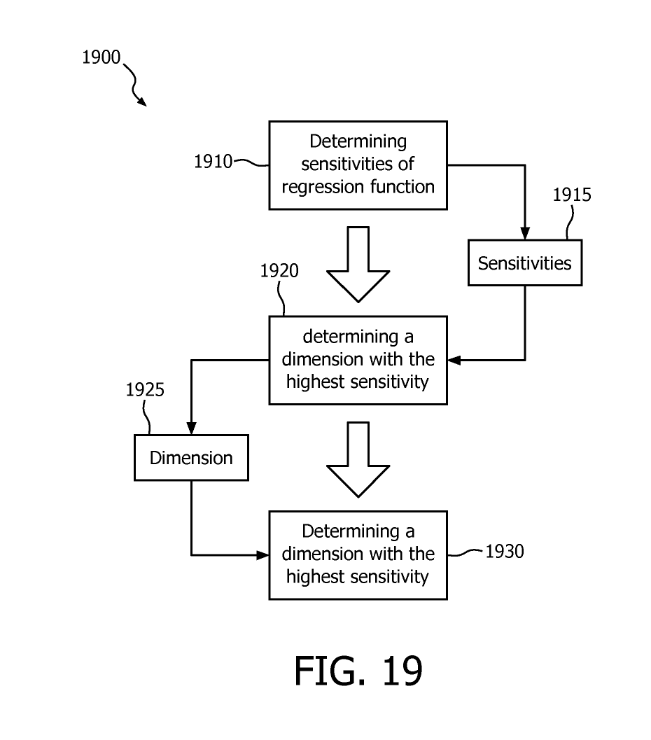

FIG. 19 shows a block diagram of a method 1900 for predicting the future pain and/or for determining the cause of the pain according to some embodiments. The method determines 1910 sensitivities 1915 of the regression function forming the predictive model 1415 at a point corresponding to the values of the physiological parameter and the activity of the user along at least some dimensions of the regression function. For example, the method 1900 determines the full gradient of the regression function.

Next, the method 1900 determines 1920 a dimension 1925 of the regression function with the highest sensitivity leading to an increase or decrease of the intensity levels of pain on the regression function and executes 1930 the action that commands to modify the values of the physiological parameter or the activity of the user corresponding to the dimension. For example, when the predicted intensity level of pain is above a threshold, the method can request the user to implement the activity that can decrease of the pain. For example, such an activity can correspond to the dimension 1925 with the highest negative value. For example, when the predicted intensity level of pain is below the threshold, the method can request the user to interrupt an activity leading to further increase of the pain. For example, such an activity can correspond to the dimension 1925 with the highest positive value.

There are several potential additional embodiments based on variations of the previously described embodiments where the predictive model is not based on the observations from the same subject but another subject with a similar pain condition, or a generic model of a user with similar conditions based on a model of pain experience.

The various methods may be performed by software, such as train microphone software 310, train predictive model software 340, execute predictive model software 345, wearable sensor software 330, etc. are software modules that are stored in the memory (of wearable device/connected device or server) and operate in conjunction with a processing device, such as controller 135/185. It should be apparent from the foregoing description that various exemplary embodiments of the invention may be implemented in hardware and/or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claim.

1. A computer implemented method for providing pain management using a wearable device, the method comprising:
determining a predictive model estimating an intensity level of pain as a function of at least one physiological parameter of a user of the wearable device and at least one activity of the user, wherein the activity of the user includes at least one of: a type of the activity, a level of the activity, a location of the activity, and a duration of the activity, wherein determining the predictive model comprises:
obtaining inputs from the user via a user interface, the inputs corresponding to the intensity level of an occurrence of pain experienced by the user;
obtaining physiological data from one or more physiological sensors;
obtaining activity data from one or more activity sensors including a combination of at least one motion sensor for determining the type of the activity and the duration of the activity and at least one sensor for determining the location of the activity;
wherein the physiological data and the activity data are taken concurrently with the occurrence of pain experienced by the user; and
correlating the intensity levels of the occurrence of pain experienced by the user with the obtained physiological data and the activity data to determine the predictive model;
determining concurrently measurements of the one or more physiological sensors of the wearable device and the one or more activity sensors of the wearable device to produce values of the physiological parameter and the activity of the user;
predicting the intensity level of pain based on the predictive model and the values of the physiological parameter and the activity of the user; and
executing one or more actions based on the predicted intensity level of pain, wherein at least some steps of the method are performed by a processor of the wearable device.

2. (canceled)

3. The method of claim 1, wherein the user interface includes a microphone for accepting auditory inputs, further comprising:
classifying the auditory inputs to determine the intensity level of the occurrence of pain experienced by the user.

4. The method of claim 1, wherein the executing comprises:
evaluating the predicted intensity level of pain with rules stored in a memory of the wearable device; and
executing one or more actions based on the evaluation of the predicted intensity level of pain with the rules.

5. The method of claim 1, wherein the determining the predictive model further comprises:
obtaining physiological data from the measurements of the physiological sensors collected for a period of time;
obtaining activity data from the measurements of the activity sensors collected for the period of time;
obtaining times and intensity levels of occurrences of pain experienced by the user within the period of time; and
determining the predictive model as a regression function correlating different intensity levels of pain with combinations of the physiological data and the activity data.

6. The method of claim 5, wherein the regression function is a multi-dimensional function, wherein a particular dimension of the regression function corresponds to values of a particular physiological parameter or a particular activity of the user.

7. The method of claim 6, wherein the predicted intensity level of pain is above a threshold, further comprising:
determining sensitivities of the regression function at a point corresponding to the values of the physiological parameter and the activity of the user along at least some dimensions of the regression function;
determining a dimension of the regression function with the highest sensitivity leading to a decrease of the intensity levels of pain on the regression function; and
executing the action that commands to modify the values of the physiological parameter or the activity of the user corresponding to the dimension.

8. The method of claim 6, wherein the predicted intensity level of pain is below a threshold, further comprising:
determining sensitivities of the regression function at a point corresponding to the values of the physiological parameter and the activity of the user along at least some dimensions of the regression function;
determining a dimension of the regression function with the highest sensitivity leading to an increase of the intensity levels of pain on the regression function to above the threshold; and
executing the action that commands to modify the values of the physiological parameter or the activity of the user corresponding to the dimension.

9. The method of claim 5, further comprising:
updating the predictive model in response to receiving the time and the intensity level of the occurrence of pain experienced by the user.

10. The method of claim 9, further comprising:
receiving a time instance and the intensity level of the occurrence of pain experienced by the user at the time instance;
retrieving a subset of the physiological data and the activity data of the user preceding the time instance; and
updating the predictive model using the subset of the physiological data and the activity data and the intensity level of the occurrence of pain experienced by the user at the time instance.

11. A wearable device for providing pain management, comprising:
a user interface configured for obtaining inputs from a user of the wearable device, each input indicates an intensity level of an occurrence of pain experienced by the user;
one or more physiological sensors to measure a value at least one physiological parameter of the user;
one or more activity sensors to determine a value of at least one activity of the user, wherein the activity of the user includes at least one of: a type of the activity and a location of the activity; and
a processor executing instructions stored in memory, wherein the processor is configured for executing the instructions to:
determine a predictive model estimating an intensity level of a pain as a function of the physiological parameter of the user of the wearable device and the activity of the user, wherein determining the predictive model comprises:
obtaining inputs from the user via a user interface, the inputs corresponding to the intensity level of an occurrence of pain experienced by the user;
obtaining physiological data from the physiological sensors;
obtaining activity data from the activity sensors including a combination of the motion sensor for determining the type of the activity and the duration of the activity and the location sensor for determining the location of the activity;
wherein the physiological data and the activity data are taken concurrently with the occurrence of pain experienced by the user; and
correlating the intensity levels of the occurrence of pain experienced by the user with the obtained physiological data and the activity data to determine the predictive model;
predict the intensity level of the pain based on the predictive model and the values of the physiological parameter and the activity of the user obtained from the physiological and the activity sensors; and
execute one or more actions based on the predicted intensity level of pain.

12. The wearable device of claim 11, wherein the user interface includes a microphone.

13. The wearable device of claim 11, wherein the processor further determines the predictive model by:
obtaining physiological data from the measurements of the physiological sensors collected for a period of time, wherein the physiological data is time-series data;
obtaining activity data from the measurements of the activity sensors collected for the period of time, wherein the activity data is time-series data;
obtaining times and intensity levels of occurrences of pain experienced by the user within the period of time; and
determining the predictive model as a regression model correlating different intensity levels of pain with a time series profile formed by a combination of the physiological data and the activity data.

14. The wearable device of claim 11, wherein at least one action executed based on the evaluation includes notifying the user by displaying a message on the wearable device.

15. A non-transitory computer-readable storage medium, having embodied thereon a program executable by a processor to perform a method for providing pain management using a wearable device, the method comprising:
determining measurements of one or more physiological sensors of the wearable device to produce a value of a physiological parameter of a user of the wearable device;
determining measurements of one or more activity sensors of the wearable device to produce a value of an activity of the user of the wearable device, wherein the activity of the user includes one or combination of a type of the activity and a location of the activity;
predicting the intensity level of pain based the values of the physiological parameter and the activity of the user and a predictive model correlating an intensity level of a pain as a function of at least one physiological parameter of the user and at least one activity of the user, wherein the predictive model is determined by:
obtaining inputs from the user via a user interface, the inputs corresponding to the intensity level of an occurrence of pain experienced by the user;
obtaining physiological data from the physiological sensors;
obtaining activity data from the activity sensors including a combination of at least one motion sensor for determining the type of the activity and the duration of the activity and at least one location sensor for determining the location of the activity;
wherein the physiological data and the activity data are taken concurrently with the occurrence of pain experienced by the user; and
correlating the intensity levels of the occurrence of pain experienced by the user with the obtained physiological data and the activity data to determine the predictive model; and
executing one or more actions based on the predicted intensity level of pain.
